(19) United States
(12) Patent Application Publication
(10) Pub. No.: US 2025/0367239 A1
(43) Pub. Date: Dec. 4, 2025

(54) COMPOSITIONS AND METHODS FOR NON-GENOTOXIC CONDITIONING

(71) Applicant: Maro Bio Inc., South San Francisco, CA (US)

(72) Inventors: Akanksha Chhabra, South San Francisco, CA (US); Vanessa Soros, South San Francisco, CA (US); Alan Le, South San Francisco, CA (US)

(21) Appl. No.: 19/107,502

(22) PCT Filed: Aug. 29, 2023
(86) PCT No.: PCT/US2023/031423

§ 371 Date: Feb. 27, 2025

Related U.S. Application Data
(60) Provisional application No. 63/401,910, filed on Aug. 29, 2022.

(51) Int. Cl. A61K 35/28; A61K 35/12; A61K 40/10; A61K 40/50; C07K 16/28; C12N 5/00; C12N 5/0789; C12N 5/10; C12N 9/22; C12N 15/113; C12N 15/864

(52) U.S. Cl. CPC A61K 35/28; A61K 40/10; A61K 40/50; C07K 16/283; C07K 16/2896; C12N 5/0647; C12N 5/10; C12N 5/562; C12N 9/226; C12N 15/113; C12N 15/8645; A61K 2035/124; C07K 2317/24; C07K 2317/31; C07K 2317/52; C07K 2317/76; C12N 2310/20; C12N 2310/321; C12N 2510/00

(57) ABSTRACT

Provided herein are methods and compositions relating to the use of anti-CD1 10 and anti-CD117 conditioning agents for depletion of endogenous hematopoietic stem cells in a subject, for example. prior to hematopoietic stem cell transplantation. Also provided are cell-based therapy methods and compositions.

### CROSS REFERENCE TO RELATED APPLICATIONS

This application claims the benefit of, and priority to, U.S. provisional patent application Ser. No. 63/401,910, filed on Aug. 29, 2022, which is hereby incorporated by reference herein in its entirety.

### FIELD

Provided herein are methods and compositions relating to the use of antibody compositions to deplete hematopoietic stem cells in a subject. The methods and compositions of the disclosure are useful, for example, for non-myeloablative conditioning prior to allogeneic and autologous hematopoietic stem cell transplantation (HSCT).

### BACKGROUND

Lifelong production of the hematopoietic cells in an individual depends on a rare population of hematopoietic stem cells that are capable of self-renewal. Because of this unique property, hematopoietic stem cell transplantation (HSCT) is a powerful therapy having the potential to correct a variety of disorders such as, but not limited to, hemoglobinopathies, autoimmune disorders and hematological malignancies. Prior to receiving an HSCT, the recipient must undergo conditioning, which serves the purposes of: (1) resetting the immune system (in the case of non-autologous transplants), (2) clearing the microenvironment, and (3) preparing bone marrow niche for donor cell engraftment, to enable reconstituting of the hematopoietic system by donor hematopoietic stem cells. Traditional conditioning regimens can involve administration of chemotherapeutic agents, irradiation, and/or immunosuppression. Because these methods are highly toxic in the short-and long-term and may trigger many life-threatening side effects, including hematological malignancies, organ damage, organ failure and infections (Gyurkocza et al. Blood (2014), 124:344-353), there exists a need for less genotoxic or non-genotoxic conditioning regimens, so that broader patient populations can be amenable to HSCT therapies that are safer while still efficacious.

Recent efforts have focused on developing conditioning regimens that lack genotoxic effects, including methodologies that utilize monoclonal antibodies that block hematopoietic stem cell survival factors, CAR T mediated-mediated conditioning, and antibody-drug conjugates (ADCs) (see, e.g., Czechowicz et al., 318 (5854) Science 1296-9 (2007); Arai et al., 26 (5) Molecular Therapy 1181-1197 (2018); and Palchaudari et al., 34 (7) Nature Biotechnology 738-745 (2016)). One such antibody-based approach targets CD117 for hematopoietic stem cell depletion. While CD117 is highly expressed on hematopoietic stem cells and progenitors, a strategy which targets CD117 alone is not sufficient to prepare an immune-competent subject for a successful hematopoietic stem cell transplant (see e.g., Xue et al, Blood 116, 5419-5422 (2010). Instead, a combination of anti-CD117 with CD47 blockade is needed (see e.g. Chhabra et al., 10:8(351) Science Translational Medicine 351ra105 (2016)), or the CD117 antibody must be combined with a toxin to promote depletion of endogenous hematopoietic stem cells and enable engraftment of donor cells (see e.g. Czechowicz et al, Nat Commun 10, 617 (2019)). Thus, there is a need for additional antibody-based conditioning regimens which can promote robust hematopoietic stem cell depletion and engraftment while substantially reducing the morbidity and mortality of HSCT.

### SUMMARY

Provided herein are methods and compositions relating to the use of anti-CD110 and anti-CD117conditioning agents, for example antibodies or antigen-binding fragments thereof, for depletion of endogenous hematopoietic stem cells in a subject, for example, prior to HSCT. Also provided are cell-based therapy methods and compositions. While not intending to be bound by any particular theory of operation, the Examples provided below demonstrate that concomitant targeting of CD110 and CD117 that are co-expressed on hematopoietic stem cells with antibodies that leverage Fc effector cell mediated clearance, results in robust and synergistic depletion of endogenous hematopoietic stem cells and engraftment of donor hematopoietic stem cells, followed by multilineage hematopoietic reconstitution in immunocompetent mice. Because this non-genotoxic conditioning occurs without the use of non-selective myeloablative conditioning agents such as irradiation or chemotherapy, concomitant targeting of CD110 and CD117 has the potential to extend the use of hematopoietic stem cell transplantation therapy to a broader spectrum of patients across a diversity of diseases and conditions.

Accordingly, in one aspect, provided herein is a method of hematopoietic stem cell engraftment in a subject in need thereof, the method comprising: (a) depleting endogenous hematopoietic stem cells in the subject by administering to the subject a pharmaceutical composition comprising: (i) a first targeting moiety that specifically binds CD117; and (ii) a second targeting moiety that specifically binds CD110; and (b) administering exogenous hematopoietic stem cells to the subject; wherein administration of the pharmaceutical composition mediates engraftment of the exogenous hematopoietic stem cells resulting in multi-lineage hematopoietic reconstitution in the subject. In some embodiments, the first and second moiety bind hematopoietic stem cells (HSCs) co-expressing CD117 and CD110. In some embodiments, the HSCs co-expressing CD117 and CD110 are long-term hematopoietic stem cells (LT-HSCs).

In some embodiments, the first targeting moiety comprises an isolated antibody or an antigen-binding fragment thereof that specifically binds CD117. In some embodiments, the isolated antibody or antigen-binding fragment thereof that specifically binds CD117 functionally disrupts signaling between Stem Cell Factor (SCF) and CD117 and/or mediates clearance of CD117 expressing cells via Fc effector function. In some embodiments, the second targeting moiety comprises an isolated antibody, or an antigen-binding fragment thereof, that specifically binds CD110. In some embodiments, the isolated antibody or antigen-binding fragment thereof that specifically binds CD110 functionally disrupts signaling between Thrombopoietin (TPO) and CD110 and/or mediates clearance of CD110 expressing cells via Fc effector function. In some embodiments, the isolated antibody of the first and/or second targeting moiety is a monoclonal antibody. In some embodiments, the antigen binding fragment of the first and/or second targeting moiety is selected from the group consisting of a Fv fragment, Fab fragment, F(ab′)2 fragment, Fab′ fragment, scFv (sFv) fragment, scFv-Fc fragment, single-chain Fvs (scFv), single-chain antibody, disulfide-linked Fvs (dsFv), fragments comprising either a V_{L} or V_{H} domain, a heavy chain antibody (hcAb), a single domain antibody (sdAb), a minibody, and a variable domain derived from camelid heavy chain antibodies (VHH or nanobody). In some embodiments, both the first targeting moiety and the second targeting moiety are comprised on the same antibody or antigen binding fragment thereof. In some embodiments, the antibody or antigen binding fragment thereof is selected from the group consisting of a diabody, diabody-Fc, single-chain diabody, tandem diabody (Tandab's), tandem scFv, tandem scFv-scFc, tandem di-scFvs, tandem tri-scFvs, multivalent antibody, bivalent or bispecific single chain variable fragment, bispecific IgG and Fab-IgG bispecific. In some embodiments, the isolated antibody or antigen binding fragment of the first and/or second targeting moiety comprises an Fc region capable of binding the neonatal Fc receptor (FcRn) of the subject. In some embodiments, the isolated antibody or antigen binding fragment of the first and/or second targeting moiety is chimeric, humanized, or human. In some embodiments, the isolated antibody or antigen binding fragment of the first and/or second targeting moiety comprises a human Fc region.

In another aspect, provided herein is a method of hematopoietic stem cell engraftment in a subject in need thereof, the method comprising: (a) depleting endogenous hematopoietic stem cells in the subject by co-administering to the subject: (i) an effective amount of a first isolated antibody, or an antigen-binding fragment thereof, that specifically binds CD117; and (ii) an effective amount of a second isolated antibody, or an antigen-binding fragment thereof, that specifically binds CD110; and (b) administering exogenous hematopoietic stem cells to the subject; wherein co-administration of the effective amounts of the first and second antibodies, or fragments thereof, synergistically mediates engraftment of the exogenous hematopoietic stem cells resulting in multi-lineage hematopoietic reconstitution in the subject. In some embodiments, the first and second isolated antibody, or antigen binding fragments thereof, bind hematopoietic stem cells co-expressing CD117 and CD110. In some embodiments, the HSCs co-expressing CD117 and CD110 are LT-HSCs.

In some embodiments, the first isolated antibody, or antigen-binding fragment thereof, functionally disrupts signaling between Stem Cell Factor (SCF) and CD117 and/or mediates clearance of CD117 expressing cells via Fc effector function. In some embodiments, the second isolated antibody, or antigen-binding fragment thereof, functionally disrupts signaling between Thrombopoietin (TPO) and CD110 and/or mediates clearance of CD110 expressing cells via Fc effector function. In some embodiments, the first isolated antibody and/or the second isolated antibody is a monoclonal antibody. In some embodiments, the first isolated antibody and/or the second isolated antibody is a bispecific antibody. In some embodiments, the antigen binding fragment that specifically binds CD117 is selected from the group consisting of a Fv fragment, Fab fragment, F(ab′)2 fragment, Fab′ fragment, scFv (sFv) fragment, scFv-Fc fragment and nanobody fragment. In some embodiments, the antigen binding fragment that specifically binds CD110 is selected from the group consisting of a Fv fragment, Fab fragment, F(ab′)2 fragment, Fab′ fragment, scFv (sFv) fragment, scFv-Fc fragment, and nanobody fragment. In some embodiments, the Fc region of the first isolated antibody and/or the second isolated antibody is capable of binding the neonatal Fc receptor (FcRn) of the subject. In some embodiments, the first isolated antibody or antigen-binding fragment thereof and/or the second isolated antibody or antigen-binding fragment thereof is chimeric, humanized, or human. In some embodiments, the first isolated antibody or antigen-binding fragment thereof and/or the second isolated antibody or antigen-binding fragment thereof comprises a human Fc region. In some embodiments, the subject is human.

In some embodiments, the antibody or antigen-binding fragment thereof that specifically binds CD117 and/or the antibody or antigen-binding fragment thereof that specifically binds CD110 is conjugated to a toxin. In some embodiments, the toxin is selected from the group consisting of saporins, saporin derivatives, ricin, abrin, gelonin, momordin, apitoxin, shiga toxins, shiga-like toxins, T-2 mycotoxin, diphtheria toxin, busulfan, pseudomonas exotoxin A, Ricin A chain derivatives, trichosanthin, luffin toxin, maytansine, amatoxin, mechlorethamine, cyclophosphamide, ethylenimine, methylmelamine, methotrexate, fluorouracil, floxuridine, cytarabine, mercaptopurine, azathioprine, thioguanine, fludarabine phosphate, cladribine, dolastatin, auristatin, auristatin E, auristatin F, MMAF, MMAE, MMAD, DMAF, or DMAE, maytansine, DM1 or DM4, duocarmycin, calicheamicin, pyrrolobenzodiazepine, exatecan, and any combination thereof.

In some embodiments, the methods provided herein further comprise monitoring the subject for depletion of endogenous hematopoietic stem cells prior to administering exogenous hematopoietic stem cells. In some embodiments, the exogenous hematopoietic stem cells are administered to the subject after the first and second targeting moieties, or first and second isolated antibodies or antigen-binding fragment(s) thereof, have substantially cleared from the blood of the subject. In some embodiments, the administering of exogenous hematopoietic stem cells to the subject occurs within 3, 5, 7 or 10 days of co-administering the the first and second targeting moieties, or the first and second isolated antibodies or antigen-binding fragment(s) thereof, to the subject.

In some embodiments, the exogenous hematopoietic stem cells are allogeneic hematopoietic stem cells. In some embodiments, the exogenous hematopoietic stem cells are autologous hematopoietic stem cells. In some embodiments, the exogenous hematopoietic stem cells comprise CD34+ hematopoietic stem and progenitor cells (HSPCs). In some embodiments, the CD34+ HSPCs comprise CD34+/CD38−/CD90+ HSPCs. In some embodiments, the CD34+ HSPCs comprise CD34+/CD38−/CD90+/CD45RA-HSPCs.

In some embodiments, the methods provided herein further comprise one or more of the following steps: (a) collecting a population of hematopoietic stem cells from the subject prior to depletion; (b) culturing the collected population of hematopoietic stem cells; and (c) cryopreserving the collected population of hematopoietic stem cells. In some embodiments, collecting the population of hematopoietic stem cells from the subject comprises one or more of the following steps: (i) mobilizing the population of hematopoietic stem cells; and (ii) collecting the population of hematopoietic stem cells by apheresis.

In some embodiments, the exogenous hematopoietic stem cells are genetically modified. In some embodiments, the exogenous hematopoietic stem cells are genetically modified using one or more components of a gene editing system. In some embodiments, the one or more components of the gene editing system is selected from the group consisting of: (i) a CRISPR/Cas guide RNA, (ii) a DNA molecule encoding a CRISPR/Cas guide RNA, (iii) a nucleic acid molecule encoding a CRISPR/Cas RNA-guided polypeptide, (iv) a CRISPR/Cas RNA-guided polypeptide, (v) a CRISPR/Cas guide RNA complexed with a CRISPR/Cas RNA-guided polypeptide, (vi) a nucleic acid molecule encoding a zinc finger protein (ZFP), (vii) a ZFP, (viii) a nucleic acid molecule encoding a transcription activator-like effector (TALE) protein, (ix) a TALE protein, and (x) a DNA donor polynucleotide. In some embodiments, the CRISPR/Cas RNA-guided polypeptide is a base editor or a prime editor. In some embodiments, the one or more components of the gene editing system comprises a nuclease capable of generating a double-strand break within a gene locus of a cell. In some embodiments, the one or more components of the gene editing system further comprises a DNA donor polynucleotide. In some embodiments, the DNA donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR).

In some embodiments, the gene editing system comprises a CRISPR nuclease and a single guide RNA (sgRNA) capable of hybridizing to a target sequence within the gene locus, wherein the sgRNA guides the CRISPR nuclease to the target sequence. In some embodiments, the CRISPR nuclease is a Cas protein. In some embodiments, the Cas protein is Cas9 or a high-fidelity variant thereof. In some embodiments, the sgRNA and the CRISPR nuclease are formed in a ribonucleoprotein (RNP) complex. In some embodiments, the sgRNA comprises one or more chemically modified nucleotides. In some embodiments, the modified nucleotide is selected from the group consisting of: a 2′-O-methyl nucleotide, a 2′-O-methyl 3′-phosphorothioate nucleotide, and a 2′-O-methyl 3′-thioPACE nucleotide. In some embodiments, a 5′ end, a 3′ end, or a combination thereof of the modified sgR.NA comprises a modified nucleotide. In some embodiments, the method further comprises contacting the population of stem cells with an AAV vector comprising a donor polynucleotide sequence. In some embodiments, the genetic modification corrects a gene mutation, replaces a mutant allele with a wild-type allele, or inserts a nucleic acid sequence encoding a therapeutic protein.

In some embodiments, the subject suffers from a disease. In some embodiments, the disease is a hemoglobinopathy. In some embodiments, the hemoglobinopathy is selected from the group consisting of sickle cell disease, α-thalassemia, β-thalassemia, and δ-thalassemia.

In another aspect, provided herein is a method of depleting endogenous hematopoietic stem cells in a subject in need thereof, the method comprising administering to the subject a pharmaceutical composition comprising: (a) a first targeting moiety that specifically binds CD117; and (b) a second targeting moiety that specifically binds CD110. In some embodiments, administration of the pharmaceutical composition mediates depletion of the exogenous hematopoietic stem cells in the subject. In some embodiments, the first and second moiety bind hematopoietic stem cells co-expressing CD117 and CD110. In some embodiments, the hematopoietic stem cells co-expressing CD117 and CD110 are LT-HSCs.

In another aspect, provided herein is a method of depleting endogenous hematopoietic stem cells in a subject in need thereof, the method comprising co-administering to the subject: (a) an effective amount of a first isolated antibody, or an antigen-binding fragment thereof, that specifically binds CD117; and (b) an effective amount of a second isolated antibody, or an antigen-binding fragment thereof, that specifically binds CD110. In some embodiments, co-administration of the effective amounts of the first and second antibodies, or fragments thereof, synergistically mediates depletion of the exogenous hematopoietic stem cells in the subject.

In another aspect, provided herein are compositions and kits comprising an antibody, or an antigen-binding fragment thereof, that specifically binds CD117; an antibody, or an antigen-binding fragment thereof, that specifically binds CD110; hematopoietic stem cells, and/or instructions for their preparation or use according to the methods described herein. The compositions, kits, and methods described herein can be used, for example, for the treatment of cancers, autoimmune disorders, viral diseases, and hematological diseases and for inducing tolerance.

### BRIEF DESCRIPTION OF THE FIGURES

FIG. 1 depicts sensorgrams demonstrating binding of antibodies to murine CD110 or murine CD117 as measured by biolayer interferometry (ForteBio Octet). (A) anti-mCD110 antibody binds to recombinant mouse CD110 extracellular domain (ECD) and (B) anti-mCD117 antibody binds to recombinant mouse CD117 (ECD).

FIG. 2 depicts a summary schematic of the study design protocol for conditioning of recipients with anti-mCD117 and anti-mCD110 antibodies.

FIG. 3 depicts (A) total chimerism of donor-derived hematopoietic cells in peripheral blood at 4, 8, 12, and 16 post-transplant following antibody-based conditioning. Donor-derived blood chimerism of (B) Gr-1⁺ Mac-1⁺ myeloid cells, (C) CD19⁺ B cells, (D) CD3⁺ T cells and (E) NK1.1+ NK cells.

FIG. 4 depicts total chimerism of donor-derived hematopoietic cells in bone marrow at 16 weeks post-transplant. Donor-derived bone marrow chimerism of (A) Lin-CD117+Sca1+ (“LSK”) cells, (B) Lin⁻CD117 ⁻Sca1⁺SLAM⁺Flt3⁻ (“LT-HSC”) cells, (C) common myeloid progenitor (“CMP”: Lin⁻CD117⁺Sca1⁻CD16/32⁻CD34⁺), (D) granulocyte-monocyte progenitor (“GMP”: Lin⁻CD117⁺Sca1⁻CD16/32⁺CD34⁺), (E) megakaryocyte-erythrocyte progenitor (“MEP”: Lin⁻CD117⁺Sca1⁻CD16/32⁻CD34⁻), and (F) common lymphoid progenitor (“CLP”: Lin⁻ CD117⁺Sca1⁺CD127⁺) populations.

FIG. 5 depicts donor-derived hematopoietic chimerism of Lin⁻CD117⁺Sca1⁺ (“LSK”) cells and Lin⁻CD117⁺Sca1⁺SLAM Flt3⁻ (“LT-HSC”) cells at 16 weeks post-transplant following antibody-based conditioning using antibodies with different Fc formats. (A) Chimerism of donor-derived LSK and LT-HSC populations following antibody-based conditioning in regimens in which the anti-mCD117 and anti-mCD110 antibodies had a murine Fc of G2a isotype. (B) Chimerism of donor-derived LSK and LT-HSC populations following antibody-based conditioning in regimens combining either ACK2 (anti-mCD117 rat IgG2b) and AMM2 (anti-mCD110rat IgG1) or anti-mCD110 and anti-mCD117 bearing murine IgG2a Fc with a mutation (N297A) that reduces binding to Fc gamma receptors.

FIG. 6 depicts CD117 and CD110 receptor counts in bone marrow of C57BL/6J (“B6”) mice. A) Representative gating of mouse HSPCs is shown on Lin⁻ and Lin⁻CD117⁺Sca1⁺ (“LSK”) cells. B) Median CD117 expression is shown on Lin-, LSK, LT-HSC, common myeloid progenitor (“CMP”: Lin⁻CD117⁺Sca1⁻CD16/32⁻CD34⁺), granulocyte-monocyte progenitor (“GMP”: Lin⁻CD117⁺Sca1⁻CD16/32⁺CD34⁺), megakaryocyte-erythrocyte progenitor (“MEP”: Lin⁻CD117⁺Sca1⁻CD16/32⁻CD34⁻), and common lymphoid progenitor (“CLP ”: Lin⁻CD117⁺Sca1⁺CD127⁺) populations. C) Median CD110 expression is shown on Lin⁻, LSK, LT-SC, CMP, GMP, MEP, and CLP populations.

FIG. 7 provides analysis of CD117 and CD110 expression in human bone marrow mononuclear cells isolated from bone marrow aspirates. Sample gating for assessment of LT-HSC (Lin-CD34+CD38−CD45RA-CD90+CD49f+) demonstrating expression of CD117 and CD110.

### DETAILED DESCRIPTION

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the singular forms “a,” “an,” and “the” include the plural referents unless the context clearly indicates otherwise.

The terms “about” and “approximately” indicate and encompasses an indicated value and a range above and below that value. In certain embodiments, the term “about” indicates a range within 20%, within 15%, within 10%, within 9%, within 8%, within 7%, within 6%, within 5%, within 4%, within 3%, within 2%, within 1%, or less of a given value or range. In certain embodiments, the term “about” indicates the designated value ±one standard deviation of that value.

The term “combinations thereof” includes every possible combination of elements to which the term refers to.

The terms “CD110,” “c-MPL” and “MPL” are used interchangeably herein. CD110 is also known by synonyms, including thrombopoietin receptor and myeloproliferative leukemia protein, among others. Unless specified otherwise, the terms include any variants, isoforms and species homologs of human CD110 that are naturally expressed by cells, or that are expressed by cells transfected with a c-MPL gene. CD110 proteins include, for example, human CD110 (NCBI Reference Sequence: NP_005364.1). c-MPL genes include, for example, Homo sapiens MPL proto-oncogene, thrombopoietin receptor (MPL), RefSeqGene (LRG_510) on chromosome 1 (NCBI Reference Sequence: NG_007525.1).

The terms “CD117” and “c-KIT” are used interchangeably herein. CD117 is also known by synonyms, including tyrosine-protein kinase KIT and mast/stem cell growth factor receptor (SCFR), among others. Unless specified otherwise, the terms include any variants, isoforms and species homologs of human CD117 that are naturally expressed by cells, or that are expressed by cells transfected with a c-KIT gene. CD117 proteins include, for example, human CD117 (NCBI Reference Sequence: NP_000213.1; and NP_001087241.1). c-KIT genes include, for example, Homo sapiens KIT proto-oncogene, receptor tyrosine kinase (KIT), RefSeqGene (LRG_307) on chromosome 4 (NCBI Reference Sequence: NG_007456.1).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See. e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. An antibody as described herein may be monospecific, bi-specific, or multispecific. Multispecific antibodies may be specific for different epitopes of one target polypeptide or may contain antigen-binding domains specific for more than one target polypeptide. See, e.g., Tutt et al., (1991), J. Immunol. 147:60-69; Kufer et al., (2004), Trends Biotechnol. 22:238-244; and Brinkmann and Kontermann, (2017), MABS, 9(2):182-212. The anti-CD110 antibodies and/or anti-CD117 antibodies described herein can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. In some embodiments, a bi-or multi-specific antibody described herein comprises binding specificities for both CD110 and CD117. In some embodiments, a multispecific antibody described herein comprises binding specificities for CD110 and CD117.

An “antibody fragment” comprises a portion of an intact antibody, such as the antigen binding or variable region of an intact antibody. Antibody fragments include, for example, Fv fragments, Fab fragments, F(ab′)₂ fragments, F(ab′) fragments, scFv (sFv) fragments, scFv-Fc fragments and nanobody fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂ ” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994).

“scFv-Fc” fragments comprise an scFv attached to an Fc domain. For example, an Fc domain may be attached to the C-terminus of the scFv. The Fc domain may follow the V_{H} or V_{L} . depending on the orientation of the variable domains in the scFv (i.e., V_{H}V_{L} or V_{L}V_{H}). Any suitable Fc domain known in the art or described herein may be used. In some cases, the Fc domain comprises an IgG1 Fc domain.

“Nanobody” fragments comprise only the variable domain of the heavy chain and lack a light chain and heavy chain constant domain. In some cases, the nanobody can be conjugated to other nanobodies and/or proteins to make a multispecific protein.

Antibodies described herein may also comprise additional antibody variants, such as diabodies, diabody-Fc, single-chain diabodies, tandem diabodies (Tandab's), tandem scFv, tandem scFv-scFc, tandem di-scFvs, tandem tri-scFvs, “multivalent antibodies” (e.g. trivalent or tetravalent antibodies), bivalent or bispecific single chain variable fragments, including bispecific IgG and Fab-IgG bispecific. Bis-scFv or di-scFv variants can be engineered by linking two scFv molecules with a linker. Bispecific antibodies may comprise two scFv molecules having different binding specificities ((scFv)2). Ligation can be performed by creating a single peptide chain with two VH and two VL regions, resulting in a tandem scFv (see, eg, Kufer P. et al. (2004) Trends in Biotechnology 22(5):238-244). Diabodies can be generated with scFv molecules having linker peptides that are too short for the two variable regions to fold together (eg, about 5 amino acids), forcing the scFv to dimerize. See, eg, Hollinger, Philipp et al. (July 1993) Proceedings of the National Academy of Sciences of the United States of America 90(14): 6444-8). Successfully purified multi-target affinity agents can be screened using a variety of in vitro and in vivo methods. Binding assays with engineered cell lines overexpressing CD110 or CD117 alone or in variable combinations can be used to screen for a multitarget affinity agent that favorably bind to cells expressing CD110 and CD117. Cells can be incubated with multitarget affinity agents, followed by a fluorescently labelled secondary antibody. Flow cytometry can be used to detect the level of antibody binding to the engineered cells. The multitarget affinity agents are expected to bind favorably to cells co-expressing both CD117 and CD110 concurrently, confirming their bispecific nature. The engineered cell lines can be tracked with flow cytometry if they are labeled using a variety of methods, for example, co-expression of a fluorescent protein (GFP, YFP, EBFP, etc.) along with CD110 and CD117. Alternatively, cells overexpressing the target receptors can be individually stained using CellTrace proliferation dyes to label and monitor binding of multitarget affinity agents. In addition to engineered cell lines, multitarget affinity agents can be tested against primary cells with known levels of target receptors to confirm binding against relevant cell types.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., CD110 or CD117) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule. In some embodiments, “selectively binds” refers to the ability of a selective binding compound, for example an antibody or an antigen binding fragment thereof, to bind to a target protein, such as, for example, CD110 or CD117, with greater affinity than it binds to a non-target protein. In certain embodiments, specific binding refers to binding to a target with an affinity that is at least 10, 50, 100, 250, 500, 1000 or more times greater than the affinity for a non-target.

As used herein, to “functionally disrupt” or a “functional disruption” of signaling between a stem cell surface receptor (e.g. CD110 or CD117) and its cognate ligand (e.g. thrombopoietin or stem cell factor, respectively) means that the interaction between the receptor and ligand is decreased such that the normal biological activity (e.g. hematopoietic stem cell proliferation) otherwise resulting from their interaction is attenuated. In some embodiments. the normal biological activity is eliminated. In some embodiments, functional disruption is effected by an antibody or antigen-binding fragment thereof that binds to the receptor or the ligand and blocks or dampens binding of the ligand to the receptor, and/or antagonizes the function of the ligand or the receptor such that normal signaling between the ligand and receptor cannot be achieved. In other embodiments, the functional disruption is achieved by a mechanism other than direct binding or direct inhibition of the receptor or the ligand. For example, the functional disruption may be achieved by binding and/or inhibiting a cofactor, upstream signaling molecule, or downstream signaling molecule to the receptor or ligand which may, for example, be required for effective signaling between the ligand and receptor. In some embodiments, functional reduction means that binding or signaling between the receptor and its cognate ligand is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to the signaling between the receptor and ligand under physiological conditions. Any method known in the art useful for assessing biological activity resulting from signaling between the receptor and its cognate ligand can be used to assess the functional disruption, including but not limited to, cellular proliferation assays and receptor competition assays. In other embodiments of the methods provided herein, binding of a target protein by antibody or antigen-binding fragment thereof does not functionally disrupt signaling, but instead facilitates immune-mediated depletion of such antibody-bound cells, for example, through ADCC, ADCP, or CDC.

As used herein, the term “synergistic” with reference to, for example, depletion of endogenous hematopoietic stem cells and/or engraftment of exogenous hematopoietic stem cells in a subject, refers to a combination of conditioning agents described herein (e.g., use of an anti-CD110 antibody and an anti-CD117 antibody) which is more effective than the additive effects of the single conditioning agents. For example, a synergistic effect of a combination of antibodies permits the use of lower dosages of one or more of the antibodies and/or less frequent administration of said antibodies to a subject. The ability to utilize lower dosages of antibodies and/or to administer said antibodies less frequently reduces the toxicity associated with the administration of said conditioning agents to a subject without reducing the efficacy of said conditioning agents in the depletion of endogenous hematopoietic stem cells and engraftment of exogenous hematopoietic stem cells. In addition, a synergistic effect can result in improved efficacy of ensuing HSCT therapy in the prevention, management, treatment or amelioration of a given disease, such as a hemoglobinopathy. Moreover, synergistic effects of a combination of conditioning agents may avoid or reduce adverse or unwanted side effects associated with the use of any single conditioning agent.

As used herein, the terms “subject”, “individual” or “patient” refer, interchangeably, to a warm-blooded animal such as a mammal. In particular embodiments, the term refers to a human. A subject may have, be suspected of having, or be predisposed to, a disease or disorder (e.g. a hemoglobinopathy) for which receiving an HSCT may be beneficial. The term also includes livestock, pet animals, or animals kept for study, including horses, cows, sheep, poultry, pigs, cats, dogs, zoo animals, goats, primates (e.g. cynomolgus macaques, or rhesus macaques), and rodents (e.g. mice and rats). A “subject in need thereof” refers to a subject that has one or more symptoms of, that has received a diagnosis, or that is suspected of having or being predisposed to a disease or condition which may be treated with, and/or may potentially benefit from HSCT as described herein.

The term “administering” as used herein refers to a method of giving a dosage of a composition (e.g., an antibody and/or cell therapy composition) to a subject. The method of administration can vary depending on various factors (e.g., the pharmaceutical composition being administered, and the severity of the condition, disease, or disorder being treated).

The term “treating” or “treatment” refers to any one of the following: ameliorating one or more symptoms of a disease or condition; preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

An “effective amount” refers to an amount of a compound or composition, as disclosed herein effective to achieve a particular biological, therapeutic, or prophylactic result. Such results include, without limitation, the depletion of hematopoietic stem cells, the engraftment of exogenous hematopoietic stem cells, and the treatment of a disease or condition disclosed herein as determined by any means suitable in the art.

### Methods of Depleting Endogenous Hematopoietic Stem Cells

Provided herein are methods and compositions which utilize selective non-genotoxic conditioning agents for depletion of endogenous hematopoietic stem cells from bone marrow niche prior to HSCT. As described herein, ablation of endogenous hematopoietic stem cells can be achieved by concomitantly targeting CD110 and CD117 with selective antibodies or antibody fragments (“anti-CD110 and anti-CD117 conditioning agents”). Following ablation, and after substantial clearance of the conditioning agents from the recipient's circulation, exogenous donor hematopoietic stem cells can be introduced to occupy the same niche as the ablated endogenous hematopoietic stem cells. Anti-CD110 and anti-CD117 conditioning agents useful for the methods provided herein are described in detail below. In certain embodiments, the conditioning regimen does not comprise the use of high-dose non-selective myeloablative agents, such as radiation or chemotherapy, and optimally avoids their accompanying toxicities including myelosuppression, mucositis, and organ and tissue toxicity (e.g. on cells of the gastrointestinal system, hair growth), as well as risk of secondary malignancies. In particular, the compositions and methods of the disclosure combine non-genotoxic selective ablation of endogenous hematopoietic stem cells with the administration of exogenous donor hematopoietic stem cells (for example, genetically modified hematopoietic stem cells) to the recipient, which may facilitate efficient, long-term engraftment, multi-lineage hematopoietic reconstitution and immunocompetence.

### Anti-CD110 and anti-CD117 Conditioning Agents

### CD110

CD110 (c-MPL), also known as the thrombopoietin receptor, is a mediator of thrombopoietin signaling and plays a critical role in maintaining the population of quiescent long term hematopoietic stem cells in bone marrow niche. Thrombopoietin—CD110 signaling stimulates megakaryopoiesis and platelet production and directly regulates hematopoietic stem cells proliferation, as both thrombopoietin and CD110 knockout mice exhibit a severe loss of hematopoietic stem cells. See, e.g., Solar et al., Blood, 92 (1998), pp. 4-10; Yoshihara et al., Cell Stem Cell, 1 (2007), pp. 685-697; Qian et al., Cell Stem Cell, 1 (2007), pp. 671-684; and Nakamura-shizu and Suda, Ann. N.Y. Acad. Sci. 1466 (2020), pp. 51-58.

Useful anti-CD110 conditioning agents for the practice of the methods provided herein include targeting moieties, antibodies and antigen-binding fragments thereof that specifically bind CD110. In some embodiments, useful anti-CD110 targeting moieties, antibodies and antigen-binding fragments thereof are capable of functionally disrupting thrombopoietin—CD110 signaling. In other embodiments, useful anti-CD110 targeting moieties, antibodies and antigen-binding fragments thereof do not functionally disrupt thrombopoietin—CD110 signaling. In some embodiments, the anti-CD110 conditioning agent is an isolated monoclonal antibody that specifically binds CD110. In some embodiments, the anti-CD110 conditioning agent is an isolated bispecific antibody that specifically binds CD110 and also specifically binds a second antigen. In some embodiments, the second antigen is CD117. In some embodiments, the anti-CD110 conditioning agent is an isolated antigen-binding fragment that specifically binds to CD110. In some embodiments, the isolated antigen-binding fragment that specifically binds to CD110 is selected from the group consisting of an Fv fragment, Fab fragment, F(ab′)2 fragment, Fab′ fragment, scFv (sFv) fragment, and scFv-Fc fragment. In some embodiments, the antibody or antigen binding fragment thereof that specifically binds CD110 is selected from the group consisting of a diabody, diabody-Fc, single-chain diabody, tandem diabody (Tandab's), tandem scFv, tandem scFv-scFc, tandem di-scFvs, tandem tri-scFvs, multivalent antibody, bivalent or bispecific single chain variable fragment, bispecific IgG, Fab-IgG bispecific, single-chain Fvs (scFv), single-chain antibodies, disulfide-linked Fvs (dsFv), fragments comprising either a VL or VH domain, a heavy chain antibody (hcAb), a single domain antibody (sdAb), a minibody, and a variable domain derived from camelid heavy chain antibodies (VHH or nanobody). Further useful antibody or antigen binding fragment formats include those described by Wilkinson & Hale (2022), mAbs, 14:1, DOI: 10.1080/19420862.2022.2123299. Suitable anti-CD110 conditioning agents include fully human, humanized or chimeric antibodies that specifically bind CD110. Humanized antibodies are especially useful for in vivo applications in humans due to their low antigenicity. Similarly, caninized, felinized, murine etc. antibodies are especially useful for applications in dogs, cats, and other species respectively.

In particular embodiments, the anti-CD110 conditioning agent is an anti-CD110 antibody or antigen-binding fragment thereof comprising an Fc domain capable of binding the neonatal Fc receptor (FcRn) of the host species. FcRn functions as a recycling or transcytosis receptor that is responsible for maintaining IgG and albumin in the circulation, and bidirectionally transporting these two ligands across polarized cellular barriers. Accordingly, binding of the Fc domain of the anti-CD110 antibody to the FcRn of the recipient can confer similar pharmacodynamics and half-life to the anti-CD110 antibody as that of a native immunoglobulin (IgG) of the recipient. In some embodiments, similar effector function is also conferred, such as ADCC and ADCP function and complement binding. In some such embodiments, the binding affinity of the Fc domain of the anti-CD110conditioning agent to the recipient's FcRn is at least about 50%, 55%, 60%, 65%, 70%, 75%, 80%, 85%, 90%, 95% or 99% of the binding affinity of a native immunoglobulin (IgG) of the recipient to its FcRn. In some embodiments, the anti-CD110 antibody is a human, humanized or human chimeric antibody comprising an Fc domain (e.g. a human Fc domain) capable of binding FcRn of a human recipient. In some such embodiments, the binding affinity of the Fc domain of the human, humanized or human chimeric antibody to human FcRn is within at least about 50%, 55%, 60%, 65%, 70%, 75%, 80%, 85%, 90%, 95% or 99% of the binding affinity of a native immunoglobulin (IgG) of the human recipient to its FcRn. In other embodiments, the anti-CD110 antibody is a murine, murinized or murine chimeric antibody comprising an Fc domain (e.g. a murine Fc domain) capable of binding FcRn of a recipient mouse.

In other embodiments, the anti-CD110 conditioning agent is an anti-CD110 antibody or antigen-binding fragment thereof comprising an Fc domain which has reduced binding to the FcRn of the recipient. In some such embodiments, the binding affinity of the Fc domain of the anti-CD110 conditioning agent to the recipient's FcRn is less than about 50%, 45%, 40%, 35%, 30%, 25%, 20%, 15%, 10%, 5% or 1% of the binding affinity of a native immunoglobulin (IgG) of the recipient to its FcRn. In some embodiments, the Fc domain of the anti-CD110 conditioning agent is engineered to have reduced effector function, such as reduced or ablated ADCC and ADCP function and complement binding. In some embodiments, the anti-CD110 conditioning agent is an anti-CD110 antibody or antigen-binding fragment thereof comprising an Fc domain which has reduced binding to one or more of the recipient's Fc gamma receptors.

Non-limiting examples of suitable anti-CD110 antibodies include clones mAb-1.75, mAb-1.6, and mAb-1.111 (for example as described in International Patent Publication No. WO 2011/060076, which is incorporated by reference in its entirety); MAb1.6.1 (C. Abbott, et al. Hybridoma (Larchmt), 29 (2010), pp. 103-113; and AMM2 (Yoshihara et al., Cell Stem Cell, 1 (2007), pp. 685-697; IBL—America (Immuno-Biological Laboratories)). In certain embodiments, the methods comprise the use of an anti-CD110 antibody that comprises heavy chain and light chain complementarity determining regions (CDRs) of any of these antibodies. In certain embodiments, the methods comprise the use of an anti-CD110 antibody that comprises three heavy chain CDRs and three light chain CDRs of any of these antibodies. In certain embodiments, the methods comprise the use of an anti-CD110 antibody that comprises three heavy chain CDRs, three light chain CDRs, and the framework regions of any of these antibodies. In certain embodiments, the methods comprise the use of an anti-CD110 antibody that comprises the variable heavy chain (V_{H}) and the variable light chain (V_{L}) of any of these antibodies. In certain embodiments, the anti-CD110 antibody is chimeric human. In certain embodiments, the anti-CD110 antibody is humanized. In certain embodiments, the anti-CD110 antibody is human. In some embodiments, the methods comprise the use of an anti-CD110 antibody that comprises: (1) heavy chain and light chain complementarity determining regions (CDRs) of any of the above-described anti-CD110 antibodies; and (2) a human Fc domain. In certain embodiments, the methods comprise the use of an anti-CD110 antibody that comprises: (1) three heavy chain CDRs and three light chain CDRs of any of the above-described anti-CD110 antibodies; and (2) a human Fc domain. In certain embodiments, the methods comprise the use of an anti-CD110 antibody that comprises: (1) three heavy chain CDRs, three light chain CDRs, and the framework regions of any of the above-described anti-CD110 antibodies; and (2) a human Fc domain. In certain embodiments, the methods comprise the use of an anti-CD110 antibody that comprises: (1) the variable heavy chain (V_{H}) and the variable light chain (V_{L}) of any of the above-described anti-CD110 antibodies; and (2) a human Fc domain. The anti-CD110 antibody can be of any format described herein.

In some embodiments, the anti-CD110 conditioning agent is conjugated to a toxin. Anti-CD110antibody-drug conjugates (ADCs) are internalized upon binding to CD110 and administer their toxic payload to ablate hematopoietic stem cells. In some embodiments, the toxin is selected from the group consisting of saporins, saporin derivatives, ricin, abrin, gelonin, momordin, apitoxin, shiga toxins, shiga-like toxins, T-2 mycotoxin, diphtheria toxin, busulfan, pseudomonas exotoxin A, Ricin A chain derivatives, trichosanthin, luffin toxin, maytansine, amatoxin, mechlorethamine, cyclophosphamide, ethylenimine, methylmelamine, methotrexate, fluorouracil, floxuridine, cytarabine, mercaptopurine, azathioprine, thioguanine, fludarabine phosphate, cladribine, dolastatin, auristatin, auristatin E, auristatin F, MMAF, MMAE, MMAD, DMAF, or DMAE, maytansine, DM1 or DM4, duocarmycin, calicheamicin, pyrrolobenzodiazepine, exatecan, and any combination thereof.

### CD117

CD117 (c-Kit) is highly expressed in hematopoietic stem cells, multipotent progenitors (MPP), and lineage restricted progenitors such as common myeloid progenitors (CMP), granulocyte macrophage progenitors (GMP), megakaryocyte erythroid progenitor (MEP), common lymphoid progenitors (CLP) and with its ligand, stem cell factor (SCF), is essential for hematopoiesis. When CD117 binds SCF, it forms a dimer that activates its intrinsic tyrosine kinase activity, which in turn phosphorylates and activates signal transduction molecules that propagate the signal in the cell. Signals transmitted through CD117 after interaction with SCF are critical for hematopoietic stem cell survival, proliferation, and differentiation. (see e.g. Edling and Hallberg, Int J Biochem Cell Biol. (2007), 39(11):1995-1998; and Domen and Weissman, J Exp Med. (2000), 192(12):1707-1718.

Useful anti-CD117 conditioning agents for the practice of the methods provided herein include targeting moieties, antibodies and antigen-binding fragments thereof that specifically bind CD117. In some embodiments, useful anti-CD117 targeting moieties, antibodies and antigen-binding fragments thereof are capable of functionally disrupting SCF—CD117 signaling. In other embodiments, useful anti-CD110 targeting moieties, antibodies and antigen-binding fragments thereof do not functionally disrupt SCF—CD117 signaling. In some embodiments, the anti-CD117 conditioning agent is an isolated monoclonal antibody that specifically binds CD117. In some embodiments, the anti-CD117 conditioning agent is an isolated bispecific antibody that specifically binds CD117 and also specifically binds a second antigen. In some embodiments, the second antigen is CD110. In some embodiments, the anti-CD117 conditioning agent is an isolated antigen-binding fragment that specifically binds to CD117. In some embodiments, the isolated antigen-binding fragment that specifically binds to CD117 is selected from the group consisting of an Fv fragment, Fab fragment, F(ab′)2 fragment, Fab′ fragment, scFv (sFv) fragment, and scFv-Fc fragment. In some embodiments, the antibody or antigen binding fragment thereof that specifically binds CD117 is selected from the group consisting of a diabody, diabody-Fc, single-chain diabody, tandem diabody (Tandab's), tandem scFv, tandem scFv-scFc, tandem di-scFvs, tandem tri-scFvs, multivalent antibody, bivalent or bispecific single chain variable fragment, bispecific IgG, Fab-IgG bispecific, single-chain Fvs (scFv), single-chain antibodies, disulfide-linked Fvs (dsFv), fragments comprising either a VL or VH domain, a heavy chain antibody (hcAb), a single domain antibody (sdAb), a minibody, and a variable domain derived from camelid heavy chain antibodies (VHH or nanobody). Further useful antibody or antigen binding fragment formats include those described by Wilkinson & Hale (2022), mAbs, 14:1, DOI: 10.1080/19420862.2022.2123299. Suitable anti-CD117 conditioning agents include fully human, humanized or chimeric antibodies that specifically bind CD117. Humanized antibodies are especially useful for in vivo applications in humans due to their low antigenicity. Similarly, caninized, felinized, murine etc. antibodies are especially useful for applications in dogs, cats, and other species respectively.

In particular embodiments, the anti-CD117 conditioning agent is an anti-CD117 antibody or antigen-binding fragment thereof comprising an Fc domain capable of binding the neonatal Fc receptor (FcRn) of the host species. FcRn functions as a recycling or transcytosis receptor that is responsible for maintaining IgG and albumin in the circulation, and bidirectionally transporting these two ligands across polarized cellular barriers. Accordingly, binding of the Fc domain of the anti-CD117 antibody to the FcRn of the recipient can confer similar pharmacodynamics and half-life to the anti-CD117 antibody as that of a native immunoglobulin (lgG) of the recipient. In some embodiments, similar effector function is also conferred, such as ADCC and ADCP function and complement binding. In some such embodiments, the binding affinity of the Fc domain of the anti-CD117conditioning agent to the recipient's FcRn is at least about 50%, 55%, 60%, 65%, 70%, 75%, 80%, 85%, 90%, 95% or 99% of the binding affinity of a native immunoglobulin (IgG) of the recipient to its FcRn. In some embodiments, the anti-CD117 antibody is a human, humanized or human chimeric antibody comprising an Fc domain (e.g. a human Fc domain) capable of binding FcRn of a human recipient. In some such embodiments, the binding affinity of the Fc domain human, humanized or human chimeric antibody to human FcRn is within at least 20%, 25%, 30%, 35%, 40%, 45%, 50%, 55%, 60%, 65%, 70%, 75%, 80%, 85%, 90%, 95% or 99% of the binding affinity of a native immunoglobulin (IgG) of the human recipient to its FcRn. In other embodiments, the anti-CD117 antibody is a murine, murinized or murine chimeric antibody comprising an Fc domain (e.g. a murine Fc domain) capable of binding FcRn of a recipient mouse.

In other embodiments, the anti-CD117 conditioning agent is an anti-CD117 antibody or antigen-binding fragment thereof comprising an Fc domain which has reduced binding to the FcRn of the recipient. In some such embodiments, the binding affinity of the Fc domain of the anti-CD117conditioning agent to the recipent's FcRn is less than about 50%, 45%, 40%, 35%, 30%, 25%, 20%, 15%, 10%, 5% or 1% of the binding affinity of a native immunoglobulin (IgG) of the recipient to its FcRn. In some embodiments, the Fc domain of the anti-CD117 conditioning agent is engineered to have reduced effector function, such as reduced or ablated ADCC and ADCP function and complement binding. In some embodiments, the anti-CD117 conditioning agent is an anti-CD117 antibody or antigen-binding fragment thereof comprising an Fc domain which has reduced binding to one or more of the recipient's Fc gamma receptors.

Non-limiting examples of suitable anti-CD117 antibodies include ACK-2 (see Czechowicz et al., Science (2007), 318:1296-9; eBioscience); SR-1 (Chandrasekaran et al., Hum Gene Ther. (2014)25:1013-22); and AMG 191 (Pang et al., Biol Blood Marrow Transplant. (2018), 24:S230-S1 (Abstract 313)). In certain embodiments, the methods comprise the use of an anti-CD117 antibody that comprises heavy chain and light chain CDRs of any of these antibodies. In certain embodiments, the methods comprise the use of an anti-CD117 antibody that comprises three heavy chain CDRs and three light chain CDRs of any of these antibodies. In certain embodiments, the methods comprise the use of an anti-CD117 antibody that comprises three heavy chain CDRs, three light chain CDRs, and the framework regions of any of these antibodies. In certain embodiments, the methods comprise the use of an anti-CD117 antibody that comprises the V_{H} and the V_{L} of any of these antibodies. In certain embodiments, the anti-CD117 antibody is chimeric human. In certain embodiments, the anti-CD117 antibody is humanized. In certain embodiments, the anti-CD117 antibody is human. In some embodiments, the methods comprise the use of an anti-CD117 antibody that comprises: (1) heavy chain and light chain complementarity determining regions (CDRs) of any of the above-described anti-CD117 antibodies; and (2) a human Fc domain. In certain embodiments, the methods comprise the use of an anti-CD117 antibody that comprises: (1) three heavy chain CDRs and three light chain CDRs of any of the above-described anti-CD 117 antibodies; and (2) a human Fc domain. In certain embodiments, the methods comprise the use of an anti-CD117 antibody that comprises: (1) three heavy chain CDRs, three light chain CDRs, and the framework regions of any of the above-described anti-CD117 antibodies; and (2) a human Fc domain. In certain embodiments, the methods comprise the use of an anti-CD117 antibody that comprises: (1) the variable heavy chain (VH) and the variable light chain (VL) of any of the above-described anti-CD117 antibodies; and (2) a human Fc domain. The anti-CD117 antibody can be of any format described herein.

In some embodiments, the anti-CD117 conditioning agent is conjugated to a toxin. Anti-CD117antibody-drug conjugates (ADCs) are internalized upon binding to CD117 and administer their toxic payload to ablate hematopoietic stem cells. In some embodiments, the toxin is selected from the group consisting of saporins, saporin derivatives, ricin, abrin, gelonin, momordin, apitoxin, shiga toxins, shiga-like toxins, T-2 mycotoxin, diphtheria toxin, busulfan, pseudomonas exotoxin A, Ricin A chain derivatives, trichosanthin, luffin toxin, maytansine, amatoxin, mechlorethamine, cyclophosphamide, ethylenimine, methylmelamine, methotrexate, fluorouracil, floxuridine, cytarabine, mercaptopurine, azathioprine, thioguanine, fludarabine phosphate, cladribine, dolastatin, auristatin, auristatin E, auristatin F, MMAF, MMAE, MMAD, DMAF, or DMAE, maytansine, DM1 or DM4, duocarmycin, calicheamicin, pyrrolobenzodiazepine, exatecan, and any combination thereof.

### Pharmaceutical Conditioning Agent Compositions and Dosage Forms

In some embodiments, an effective dose of each of the anti-CD110 and anti-CD117 conditioning agents of the disclosure is the dose that, when administered together, depletes endogenous hematopoietic stem cells by at least 10-fold, at least 100-fold, at least 1000-fold, at least 100,000-fold or more relative to the level of hematopoietic stem cells present in the recipient's bone marrow niche prior to the administration. The effective dose will depend on the individual and the specific conditioning agent, but will generally be at least about 50 μg/kg body weight, at least about 100 μg/kg, at least about 150 g/kg, at least about 200 μg/kg, at least about 250 μg/kg, at least about 300 μg/kg, at least about 350 μg/kg, at least about 400 μg/kg, at least about 450 μg/kg, at least about 500 μg/kg, at least about 550 μg/kg, at least about 600 g/kg, at least about 650 μg/kg, at least about 700 μg/kg, at least about 750 μg/kg, at least about 800 μg/kg, at least about 850 μg/kg, at least about 900 μg/kg, at least about 950 μg/kg, at least about 1 mg/kg, and up to about 2.5 mg/kg, up to about 5 mg/kg, up to about 7.5 mg/kg, up to about 10 mg/kg, up to about 15 mg/kg, up to about 25 mg/kg, up to about 50 mg/kg, up to about 100 mg/kg. In certain embodiments, the dose is selected from 25 mg to 1000 mg, 25 mg to 750 mg, 25 mg to 650 mg, 25 mg to 500 mg. In certain embodiments, the dose is selected from 25 mg, 50 mg, 75 mg, 100 mg, 125 mg, 150 mg, 175 mg, 200 mg, 325 mg, 500 mg, and 650 mg.

The dose of one or both conditioning agents can be administered for a period of time on a schedule deemed suitable by the person of skill to effect the desired ablation of endogenous hematopoietic stem cells. In certain embodiments, the dose is administered daily In certain embodiments, the dose is administered twice per day. In certain embodiments, the dose is administered three times per day. In certain embodiments, the dose is administered four times per day. In certain embodiments, the dose is administered daily in divided doses. In some embodiments, the dose is administered for about 1 day, about 2 days, about 3 days, about 4 days, about 5 days, about 6 days or about 7 days, 1 to 2 days, 1 to 3 days, 1 to 4 days, 1 to 5 days, 1 to 6 days, 1 to 7 days, 1 to 10 days, or more.

The anti-CD110 and anti-CD117 conditioning agents may be formulated together or separately, but are administered concomitantly. “Concomitant” and “concomitantly” as used herein refer to the administration of at least two agents to a patient either simultaneously or within a time period during which the effects of the first administered agent are still operative in the patient. For example, the concomitant administration of the second agent can occur one to two days after the first, preferably within one to seven days, after the administration of the first agent.

The anti-CD110 and anti-CD117 conditioning agents of the disclosure can be formulated for administration by any technique deemed useful to the person of skill in any composition deemed useful to the person of skill. In some embodiments, the anti-CD110 and anti-CD117 conditioning agents are formulated as pills, capsules, tablets, syrups, ampules, lozenges, powders for oral administration to an individual. In some embodiments, the conditioning agents are formulated for intravenous infusion or injection. In some embodiments, the conditioning agent is a pharmaceutical composition or single unit dosage form. Pharmaceutical compositions and single unit dosage forms provided herein comprise a prophylactically or therapeutically effective amount of one or both of the anti-CD110 and anti-CD117 conditioning agents.

### Methods of HSCT and Engraftment

### Monitoring Clearance of Conditioning Agents Prior to HISCT

In some embodiments, following co-administration of anti-CD110 and anti-CD117 conditioning agents, the recipient's bone marrow niche is cleared of endogenous hematopoietic stem cells so that exogenous donor hematopoietic stem cells can newly occupy the niche. However, to avoid inadvertent clearance of the donor cells by any residual conditioning agents remaining in the recipient, the pharmacokinetic levels of one or both conditioning agents can be monitored for clearance from the recipient's blood prior to HSCT. In some embodiments, the recipient undergoes HSCT only after one or both of the anti-CD110 and anti-CD117 conditioning agents have been substantially cleared from the recipient's circulation.

In some embodiments, an anti-CD110 or anti-CD117 conditioning agent is substantially cleared from circulation when the concentration of the conditioning agent, as assessed for example from a blood sample of the recipient, is no longer detectable using any method known in the art for measuring the presence and/or activity of a biologic in blood or serum. In some embodiments, the conditioning agent is substantially cleared from circulation when it is no longer detectable above a background threshold of an assay used to detect the conditioning agent. Any method known in the art useful for detecting antibodies, or antibody fragments, such as ELISA-based detection assays, immunoprecipitation techniques and immunoblot assays, can be used to assess clearance of the conditioning agent. In certain embodiments, serum collected from the recipient at certain time points after administration of the conditioning agents can be contacted with stem cells, for example a sample of donor hematopoietic stem cells, and binding of any conditioning agents in the serum to the stem cells can be assessed using conventional methods. In other embodiments, the contacted stem cells can be assessed for growth inhibition in the presence of the recipient's serum.

In some embodiments, upon confirmation that one or both of the anti-CD110 and anti-CD117conditioning agents are sufficiently cleared from the recipient's circulation, the recipient can be administered exogenous hematopoietic stem cells. In some embodiments, sufficient clearance is achieved when the serum levels of the conditioning agent decrease a certain fold below peak levels of the conditioning agent following administration. In some embodiments, the conditioning agent is at least 10-fold, 100-fold, 1000-fold, 10,000-fold, 100,000-fold, 1,000,000-fold or greater than 1,000,000-fold below peak levels, prior to administration of exogenous donor hematopoietic stem cells. In other embodiments, the exogenous donor hematopoietic stem cells can be administered based on known or expected pharmacokinetics of the conditioning agent. In some embodiments, the exogenous donor hematopoietic stem cells are administered within 1, 2, 3, 4, 5, 6, 7, 8, 9 10 or greater than 10 days following co-administration of the anti-CD110 and anti-CD117 conditioning agents.

### Hematopoietic Stem Cell Transplantation

In certain embodiments, following administration of conditioning antibodies and depletion of endogenous hematopoietic stem cells, the methods further comprise administering to the patient an amount of exogenous hematopoietic stem cells effective for therapy. In some embodiments of the methods provided herein, the patient is administered an amount of hematopoietic stem and progenitor cells effective for therapy. In some embodiments, the administered exogenous cells can include donor bone marrow cells, umbilical cord blood cells, hematopoietic stem and progenitor cells (HSPCs), peripheral blood CD34⁺ cells, peripheral blood CD34⁺ and CD90⁺ cells, and any combination thereof.

The hematopoietic stem cells can be any hematopoietic stem cells deemed useful by the practitioner of skill. In certain embodiments, the exogenous hematopoietic stem cells, once engrafted, are capable of reconstituting hematopoiesis in the patient. Human hematopoiesis is defined by a cell surface marker expression-based hierarchy initiated by hematopoietic stem cells that both self-renew and differentiate into multipotent progenitors, which in turn give rise to lineage-restricted progenitors, and finally terminally differentiated blood cells (Baum et a., PNAS 89, 2804-2808 (1992); Majeti et al., Cell Stem Cell 1, 635-645 (2007); Doulatov et al., Cell Stem Cell 10, 120-136 (2012)). CD34⁺ expression defines the heterogeneous HSPC population, which can be further classified as a multipotent progenitor (CD34⁺/CD38⁻/CD45RA⁻), long-term repopulating cell in xenograft mice (CD34 /CD38⁻/CD90⁺), and a population highly enriched for hematopoietic stem cells (CD34⁺/CD38⁻/CD90+/CD45RA⁻).

In certain embodiments, the hematopoietic stem cells are of any subtype or colony forming unit. In certain embodiments, the hematopoietic stem cells are colony forming unit-granulocyte-erythrocyte-monocyte-megakaryocyte cells. In certain embodiments, the hematopoietic stem cells are colony forming unit-erythrocyte cells In certain embodiments, the hematopoietic stem cells are colony forming unit-granulocyte-macrophage cells. In certain embodiments, the hematopoietic stem cells are colony forming unit-megakaryocyte cells. In certain embodiments, the hematopoietic stem cells are colony forming unit-basophil cells. In certain embodiments, the hematopoietic stem cells are colony forming unit-eosinophil cells.

The hematopoietic stem cells can be from any source deemed useful to the person of skill. In certain embodiments, the hematopoietic stem cells are from a donor. In certain embodiments, the donor is the patient. In certain embodiments, the donor is another subject of the same species, for instance another human. In certain embodiments, the hematopoietic stem cells are autologous. In certain embodiments, the hematopoietic stem cells are allogeneic. In certain embodiments, the hematopoietic stem cells are syngeneic.

The hematopoietic stem cells can be harvested by any technique deemed useful to the person of skill. In some embodiments, the donor subject is administered an hematopoietic stem cells mobilizing agent (e.g plerixafor (Mozobil®), G-CSF, GM-CSF), prior to harvest. In certain embodiments, the hematopoietic stem cells are harvested from peripheral blood. In certain embodiments, the hematopoietic stem cells are harvested from cord blood. In certain embodiments, the hematopoietic stem cells are harvested from bone marrow. In some embodiments, a population of donor cells can be obtained from a product that is collected from a subject, such as a patient or subject in need of an autologous HSCT. The product can be an apheresis product that contains a heterogeneous mixture of cells that have been collected from the subject. The heterogenous mixture of cells can contain primary cells as well as primary CD34+ cells and/or human stem cells and/or progenitor cells (HSPCs). The CD34+ cells and/or HSPCs can be isolated or separated from the other cells in order to obtain a population of stem cells. Following the separation of CD34+ HSPCs, the resulting population of stem cells are substantially free of non-CD34+ cells and are ready for subsequent genetic manipulation.

In some embodiments, the harvested hematopoietic stem cells are separated from the population of primary cells using flow cytometry. In some instances, the flow cytometry comprises fluorescence-activated cell sorting (FACS). In certain other embodiments, the harvested hematopoietic stem cells are separated from the population of primary cells using magnetic bead separation. In some instances, the magnetic bead separation comprises magnetic-activated cell sorting (MACS). In certain other embodiments, the harvested hematopoietic stem cells are separated using a device configured for hematopoietic stem cell enrichment, such as the Miltenyi Biotec CliniMACS cell manufacturing platform.

Methods for culturing or expanding primary hematopoietic stem cells are known in the art, including those described in International Patent Application No. PCT/US2022/72014, which is herein incorporated by reference in its entirety. Methods for culturing primary cells and their progeny are known, and suitable culture media, supplements, growth factors, and the like are both known and commercially available. Typically, human primary cells are maintained and expanded in serum-free conditions. Alternative media, supplements and growth factors and/or alternative concentrations can readily be determined by the skilled person and are extensively described in the literature. In some embodiments, the isolated or purified gene modified cells can be expanded in vitro according to standard methods known to those of ordinary skill in the art.

In particular embodiments, the HSCT can be performed using freshly isolated populations of cells comprising hematopoietic stem cells. In other particular embodiments, HSCT of the methods contemplated herein are performed using cryopreserved populations of cells comprising hematopoietic stem cells. Cells may be cryopreserved following harvest or isolation of hematopoietic stem cells, after culture initiation and activation, after modification (for example genetic modification), or after expansion or any process step. The freeze-thaw cycle may provide a more uniform hematopoietic stem cells composition by removing the non-hematopoietic stem cell population. The hematopoietic stem cells can be stored by any technique deemed useful to the person of skill. In certain embodiments, the harvested cells are formulated in cryopreservation media and placed in cryogenic storage units such as liquid nitrogen freezers (−195° C.) or ultra-low temperature freezers (−65° C., −80° C., or −120° C.) for long term storage of at least one month, 2 months, 3 months, 4 months, 6 months, 1 year, 2 years, 3 years, or at least 5 years. In some embodiments, thawed cells are conditioned by methods described herein.

### Genetically Modified Hematopoietic Stem Cells

The methods of HSCT described herein include transplantation of hematopoietic stem cells that are genetically modified, for example, to comprise therapeutic heterologous donor polynucleotide sequences. Donor polynucleotide sequences described herein may be incorporated within a wide variety of gene therapy constructs, e.g., to deliver a nucleic acid encoding a protein to a subject in need thereof. A vector construct refers to a polynucleotide molecule including all or a portion of a viral genome and an exogenous polynucleotide sequence. In some instances, gene transfer can be mediated by a DNA viral vector, such as an adenovirus (Ad) or adeno-associated virus (AAV). Other vectors useful in methods of gene therapy are known in the art. For example, a construct of the present disclosure can include an alphavirus, herpesvirus, retrovirus, lentivirus, or vaccinia virus. The exogenous sequences generally encode recombinant molecules to be expressed in the cells, e.g., for use in cell therapy. Processing steps of the methods can also or alternatively include all or a portion of cell washing, dilution, selection, isolation, separation, cultivation, stimulation, packaging, and/or formulation. The methods generally allow for the processing, e.g., selection or separation and/or transduction, of cells on a large scale (such as in compositions of volumes greater than or at about 50 mL).

In some embodiments, hematopoietic stem cells are genetically modified using gene editing applications which utilize site-specific nucleases for knock-out of targeted genomic sequences or knock-in of exogenous sequences, and for transferring exogenous sequences to the cells by viral transduction through the use of recombinant viral vectors. In some such embodiments, hematopoietic stem cells are collected by apheresis. enriched from the apheresis product, then cryopreserved prior to performing any gene editing method (e.g., gene knock-out, gene knock-in, gene correction). Cryopreservation may be introduced after mobilization and collection (e.g. by apheresis) of stem cells and selection for hematopoietic stem cells. Following cryopreservation, an assessment can be made on whether the threshold number of hematopoietic stem cells has been collected from the donor to proceed with the gene editing steps that follow. If a threshold number of cells has not been reached from a single round of mobilization, collection, selection and cryopreservation, subsequent rounds may be performed until the threshold number of cells has been reached. Threshold numbers of hematopoietic stem cells to be collected may vary depending on a number of factors, including but not limited to, the gene editing procedure performed (e.g., gene knock-out, gene knock-in, gene correction), the targeted gene to be edited, the mechanism by which the targeted gene is modified (e.g., homology dependent repair (HDR)), the efficiency of the editing procedure (e.g. HDR efficiency) and the therapeutic threshold for treatment of a specific disease. In some embodiments, the threshold number of hematopoietic stem cells to be collected from a donor prior to gene editing is about 1×10⁴ to 1×10⁵, 1×10⁵ to 1×10⁶, 1×10⁶ to 1×10⁷ cells/kg or more. In some embodiments, at least about 1×10⁵ to 1×10⁷ cells/kg are collected prior to gene editing. In some embodiments, at least about 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8 ×10⁶, 9×10⁶, 1×10⁷, 2×10⁷, 3×10⁷, 4×10⁷, 5×10⁷, 6×10⁷, 7×10⁷, 8×10⁷, 9×10⁷, or about 1×10⁸ hematopoietic stem cells/kg are collected prior to proceeding with gene editing of the collected cells. Once the threshold number of hematopoietic stem cells are mobilized, collected, selected for, and cryopreserved, the cells can then proceed to thaw, culture and gene editing.

In some embodiments, the gene editing utilizes a nuclease introduced to the cell that is capable of causing a double-strand break near or within a genomic target site, which may be useful for increasing the frequency of homologous recombination and HDR at or near the cleavage site. In preferred embodiments, the recognition sequence for the nuclease is present in the host cell genome only at the target site, thereby minimizing any off-target genomic binding and cleavage by the nuclease. Gene-editing nucleases useful for the methods provided herein include but are not limited to a TAL-effector DNA binding domain-nuclease fusion protein (TALEN), a site-specific recombinase (for example, serine recombinase or a tyrosine recombinase, integrase (FLP, Cre, lambda integrase) or resolvase; a transposase, a zinc-finger nuclease (ZFN), and a clustered regularly interspaced short palindromic repeats (CRISPR) and CRISPR-associated (Cas) protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Csel, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In some embodiments, genetically modified CD34+ stem cells are generated by introducing a CRISPR-associated Cas nuclease (e.g. Cas9), a guide RNA polynucleotide, and a donor polynucleotide sequence into primary CD34+ stem cells. Through introduction of these components into the cell, a double stranded break can be introduced at a specific site as directed by the guide polynucleotide sequence and the CRISPR-associated Cas9 nuclease. A donor polynucleotide containing a sequence of interest can be further introduced into the cell and through homology directed recombination, the sequence of interest can be inserted into the cell. The transfer of the donor polynucleotide sequence can be carried out by transduction. The methods for viral transfer, e.g., transduction, generally involve at least initiation of transduction by incubating in a centrifugal chamber an input composition comprising the cells to be transduced and viral vector particles containing the vector, under conditions whereby cells are transduced or transduction is initiated in at least some of the cells in the input composition, wherein the method produces an output composition comprising the transduced cells.

Methods for introducing polypeptides, nucleic acids, and viral vectors (e.g., viral particles) into a primary cell, target cell, or host cell are known in the art. Any known method can be used to introduce a polypeptide or a nucleic acid (e.g., a nucleotide sequence encoding the DNA nuclease or a modified sgRNA) into a primary cell, e.g., a human primary cell. Non-limiting examples of suitable methods include electroporation (e.g., nucleofection), viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In some embodiments the Cas nuclease can be in the form of a protein. In some embodiments, the Cas nuclease can be in the form of a plasmid, thereby allowing a cell that carries this expression construct to then express the Cas nuclease. In other embodiments, the Cas nuclease is pre-complexed with a guide RNA and introduced into the cell as a ribonucleoprotein (RNP). In some embodiments, the Cas nuclease and the guide polynucleotide sequence is introduced into the CD34+ cell through electroporation.

Introduction of the donor polynucleotide can occur through viral transduction using a delivery vector, such as adeno associated virus (AAV). AAV of any serotype or pseudotype can be used. Certain AAV vectors are derived from single stranded (ss) DNA parvoviruses that are nonpathogenic for mammals. Briefly, rep and cap viral genes that can account for 96% of the archetypical wild-type AAV genome can be removed in the generation of certain AAV vectors, leaving flanking inverted terminal repeats (ITRs) that can be used to initiate viral DNA replication, packaging and integration. Wild type AAV integrates into the human host cell genome with preferential site specificity at chromosome 19q13.3. Alternatively, AAV can be maintained episomally. At least twelve human serotypes of AAV (AAV serotype 1 (AAV-1) to AAV-12) and more than 100 serotypes from nonhuman primates have been discovered to date. Any of these serotypes, as well as any combinations thereof, may be used within the scope of the present disclosure. A serotype of the viral vector can be selected from the group consisting of AAV1, AAV2, AAV3, AAV4, AAV5, AAV6, AAV7, AAV8, and AAV9. In some embodiments, the serotype is AAV6.

In some embodiments, the viral transduction occurs within 30 minutes of the electroporation. In some embodiments, the viral transduction occurs simultaneously with the electroporation. In some embodiments, the viral transduction occurs within 1, 2, 3, 4, 5, 6, 7, 8, 9, 10, 15, 20, 25, or 30 minutes of the electroporation.

In other embodiments, hematopoietic stem cells are genetically modified using gene editing applications which utilize base editors. Base editing is a CRISPR-Cas9-based genome editing technology that allows the introduction of point mutations in the DNA without generating DSBs. Two major classes of base editors have been developed: cytidine base editors or CBEs allowing C>T conversions and adenine base editors or ABEs allowing A>G conversions (see e.g. Rees et al. (2018) Nat Rev Genet 19:770-788).

In other embodiments, hematopoietic stem cells are genetically modified using gene editing applications which utilize prime editors. Prime editors (PE) consist of nCas9 fused to a reverse transcriptase used in combination with a prime editing RNA (pegRNA, a guide RNA that includes a template region for reverse transcription). Prime editing allows introduction of insertions, deletions (indels) and 12 base-to-base conversions. Prime editing relies on the ability of a reverse transcriptase (RT), fused to a Cas nickase variant, to convert RNA sequence brought by a prime editing guide RNA (pegRNA) into DNA at the nick site generated by the Cas protein. The DNA flap generated from this process is then included or not in the targeted DNA sequence. See, e.g. Anzalone et al. (2019) Nature 576:149-157. Non-limiting examples of prime editing systems include PE1, PEI-M1, PE1-M2, PE1-M3, PE1-M6, PE1-M15, PE1-M3inv, PE2, PE3, PE3b.

In other embodiments, hematopoietic stem cells are genetically modified using gene editing applications which utilize a DNA-guided polypeptide such as Natronobacterium gregoryi Argonaute (NgAgo), an RNA-guided polypeptide (e.g., Cas9, CasX, CasY, Cpf1, and the like); a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb1 integrase, R4 integrase, lambda integrase, HK022 integrase, HP1 integrase, and the like); a resolvase and/or invertase (e.g., Gin, Hin, γδ3, Tn3, Sin, Beta, and the like); a transposon and/or a DNA derived from a transposon (e.g., bacterial transposons such as Tn3, Tn5, Tn7, Tn9, Tn10, Tn903, Tn1681, and the like; eukaryotic transposons such as Tc1/mariner super family transposons, PiggyBac superfamily transposons, hAT superfamily transposons, PiggyBac, Sleeping Beauty, Frog Prince, Minos, Himar1, and the like), and including CRISPR-transposons that direct RNA-guided transposition by natively combining the DNA integration capabilities of transposases and the target programmability of CRISPR-Cas (see eg Peters et al., Proc Natl Acad Sci USA 114:E7358-E7366 (2017); Klompe et al., Nature 571:219-225(2019); and Halpin Healy et al., Nature 577:271-274 (2020).

### Pharmaceutical Hematopoietic Stem Cell Compositions

Also provided herein are methods, compositions and kits for use of hematopoietic stem cells, for example genetically modified hematopoietic stem cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals.

In some embodiments, the pharmaceutical composition comprises a modified host cell that is genetically engineered to comprise an integrated donor sequence at a targeted gene locus of the host cell. In some embodiments, the modified host cell is genetically engineered to comprise an integrated functional donor sequence, for example, a SNP donor that corrects one or mutations in a target gene (e.g. HBB) or inserts into or replaces some or all of the mutated allele with a wild-type allele. In particular embodiments, a functional donor sequence is integrated into the translational start site of the endogenous locus of the target gene. In particular embodiments, the functional donor sequence that is integrated into the host cell genome is expressed under control of the native promoter sequence of the target gene.

In some embodiments, the pharmaceutical composition comprises a plurality of the modified host cells, and further comprises unmodified host cells and/or host cells that have undergone nuclease cleavage resulting in INDELS at the target gene locus but not integration of the donor sequence. In some embodiments, the pharmaceutical composition is comprised of at least 5% of the modified host cells comprising an integrated donor sequence. In some embodiments, the pharmaceutical composition is comprised of about 9% to 50% of the modified host cells comprising an integrated donor sequence. In some embodiments, the pharmaceutical composition is comprised of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50% or more of the modified host cells comprising an integrated donor sequence. The pharmaceutical compositions described herein may be formulated using one or more excipients to, e.g.: (1) increase stability: (2) alter the biodistribution (e.g., target the cells to specific tissues or cell types, e.g. hematopoietic stem cells); and/or (3) enhance engraftment in the recipient.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., exogenous hematopoietic stem cells) and optionally one or more pharmaceutically acceptable excipients. Pharmaceutical compositions of the present disclosure may be sterile.

Relative amounts of the active ingredient (e.g. the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof. Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Aministration

In certain embodiments, the methods comprise administering to an individual in need of treatment a composition comprising an effective amount of hematopoietic stem cells (e.g. genetically modified hematopoietic stem cells). Therapeutically effective doses of the hematopoietic stem cells can be in the range of about one million to about 200 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges. In some embodiments, the method comprises administering between 2×10⁶ and 2×10⁸ viable hematopoietic stem cells per kg of body weight.

In certain embodiments, pharmaceutical compositions comprising exogenous hematopoietic stem cells in accordance with the present disclosure may be administered at dosage levels sufficient to deliver from, e.g., about 1×10⁴ to 1×10⁵, 1×10⁵ to 1×10⁶, 1×10⁶ to 1×10⁷, or more cells to the subject, or any amount sufficient to obtain the desired therapeutic or prophylactic, effect. The desired dosage of the modified host cell pharmaceutical compositions of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6years, 7 years, 8 years, 9 years, 10 years or more than 10 years. In some embodiments, only a single dose is needed to effect treatment or prevention of a disease or disorder described herein. In other embodiments, a subject in need thereof may receive more than one dose, for example, 2, 3, or more than 3 doses of a pharmaceutical hematopoietic stem cells compositions described herein to effect treatment or prevention of the disease or disorder. The hematopoietic stem cells may be used in combination with one or more other therapeutic, prophylactic, research or diagnostic agents, or medical procedures, either sequentially or concurrently. In general, each agent will be administered at a dose and/or on a time schedule determined for that agent.

The infusion population and compositions thereof can be administered to an individual in need thereof using standard administration techniques, formulations, and/or devices. Provided are formulations and administration with devices, such as syringes and vials, for storage and administration of the compositions. Formulations or pharmaceutical composition comprising exogenous hematopoietic stem cells include those for intravenous, intraperitoneal, subcutaneous, intramuscular, or pulmonary administration. Compositions of the exogenous hematopoietic stem cells can be provided as sterile liquid preparations, e.g., isotonic aqueous solutions, suspensions, emulsions, dispersions, or viscous compositions, which may in some aspects be buffered to a selected pH. Viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the hematopoietic stem cells in a solvent, such as in admixture with a suitable carrier, diluent, or excipient such as sterile water, physiological saline, glucose, dextrose, or the like.

Exogenous hematopoietic stem cells included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously. The pharmaceutical compositions may be administered to a subject using any amount and any route of administration effective for preventing, treating, or managing a disease described herein. The exact amount required will vary from subject to subject, depending on the species, age, and general condition of the subject, the severity of the disease, the particular composition, its mode of administration, its mode of activity, and the like.

In some embodiments, following administration of exogenous donor hematopoietic stem cells, the recipient can be monitored for hematopoietic recovery, reconstitution and/or donor chimerism as indicators for successful engraftment. In some embodiments, engraftment is determined by assessing donor myeloid chimerism. In some embodiments, engraftment is determined by assessing lineage specific chimerism. In some embodiments, engraftment is determined by assessing naïve T cell production. Any method known in the art for assessing donor cell chimerism may be used with the disclosed methods (see e.g. Pinkel et al., Proc Natl Acad Sci USA (1996), 83:2934-2938). In certain embodiments, following transplantation with donor stem cells, the recipient is a chimera or mixed chimera for the donor cells. Mixed chimerism (MC) is defined as the presence of more than 5% host-derived cells on more than one occasion in the whole blood. This is further categorized into high-level MC (95%-50% donor chimerism), low-level MC (49%-10% donor chimerism), or very low-level MC (<10% donor chimerism).

### Methods of Treatment

The compositions and methods of hematopoietic stem cell depletion and engraftment provided herein may be used as part of a treatment regimen for any disease or condition for which HSCT is useful. HSCT may be used to treat a number of conditions, including congenital and acquired conditions. In some embodiments, acquired conditions treatable with HSCT include but are not limited to: (1) malignancies, including hematological malignancies such as leukemias (e.g. acute lymphoblastic leukemia (ALL), acute myeloid leukemia (AML), chronic lymphocytic leukemia (CLL), chronic myelogenous leukemia (CML)), lymphomas (e.g. Hodgkin's disease, Non-Hodgkin's lymphoma), myelomas (e.g. multiple myeloma (Kahler's disease)); solid tumor cancers (e.g. neuroblastoma, desmoplastic small round cell tumor, Ewing's sarcoma, choriocarcinoma); (2) hematologic disease, including phagocyte disorders (e.g. chronic granulomatous disease), bone marrow failure disorders (e.g. myelodysplastic syndrome, Fanconi's anemia, dyskeratosis congenita), anemias (e.g. paroxysmal nocturnal hemoglobinuria, aplastic anemia, acquired pure red cell aplasia), myeloproliferative disorders (e.g. polycythemia vera, essential thrombocytosis, myelofibrosis); (3) metabolic disorders including amyloidosis (e.g. amyloid light chain (AL) amyloidosis); (4) environmentally-induced diseases such as radiation poisoning; (5) viral diseases (e.g. HTLV, HIV); and (5) autoimmune diseases such as multiple sclerosis.

In some embodiments, congenital conditions treatable with HSCT include but are not limited to: (1) lysosomal storage disorders, including lipidoses (disorders of lipid storage, such as neuronal ceroid lipofuscinoses (e.g. infantile neuronal ceroid lipofuscinosis (INCL, Santavuori disease) and Jansky-Bielschowsky disease (late infantile neuronal ceroid lipofuscinosis)); sphingolipidoses (e.g. Niemann-Pick disease and Gaucher disease), leukodystrophies (e.g. adrenoleukodystrophy, metachromatic leukodystrophy, Krabbe disease (globoid cell leukodystrophy); mucopolysaccharidoses (e.g. Hurler syndrome (MPS I H, α-L-iduronidase deficiency), Scheie syndrome (MPS I S), Hurler-Scheie syndrome (MPS I H-S), Hunter syndrome (MPS II, iduronidase sulfate deficiency), Sanfilippo syndrome (MPS III), Morquio syndrome (MPS IV), Maroteaux-Lamy syndrome (MPS VI), Sly syndrome (MPS VII)); glycoproteinoses (e.g. Mucolipidosis II (I-cell disease), fucosidosis, aspartylglucosaminuria, alpha-mannosidosis); and Wolman disease (acid lipase deficiency); (2) immunodeficiencies, including T-cell deficiencies (e.g. ataxia-telangiectasia and DiGeorge syndrome), combined T-and B-cell deficiencies (e.g. severe combined immunodeficiency (SCID), all types), well-defined syndromes (e.g. Wiskott-Aldrich syndrome), phagocyte disorders (e.g. Kostmann syndrome, Shwachman-Diamond syndrome), immune dysregulation diseases (e.g. Griscelli syndrome, type II), innate immune deficiencies (e.g. NF-Kappa-B Essential Modulator (NEMO) deficiency (Inhibitor of Kappa Light Polypeptide Gene Enhancer in B Cells Gamma Kinase deficiency)); (3) hematologic diseases, including hemoglobinopathies (e.g. sickle cell disease, thalassemia (e.g. β thalassemia)), anemias (e.g. aplastic anemia such as Diamond-Blackfan anemia and Fanconi anemia), cytopenias (e.g. Amegakaryocytic thrombocytopenia) and hemophagocytic syndromes (e.g. hemophagocytic lymphohistiocytosis (HLH)).

In some embodiments, the disease or condition is selected from the group consisting of a hemoglobinopathy, a viral infection, X-linked severe combined immune deficiency, Fanconi anemia, hemophilia, neoplasia, cancer, amyotrophic lateral sclerosis, alpha antitrypsin deficiency, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders. In some embodiments, the hemoglobinopathy is selected from the group consisting of sickle cell disease, α-thalassemia, β-thalassemia, and δ-thalassemia.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: Co-Administration of Anti-CD110 and Anti-CD117 Conditioning Agents Results in Robust Engraftment of Transplanted HSPCs

This example provides results demonstrating that in immunocompetent recipient mice, treatment with a combination of effector-competent anti-CD117 and anti-CD110 monoclonal antibodies synergize to enable robust engraftment of donor HSPCs (hematopoietic stem and progenitor cells) and multi-lineage reconstitution of hematopoietic cells.

### Materials and Methods

### Antibodies

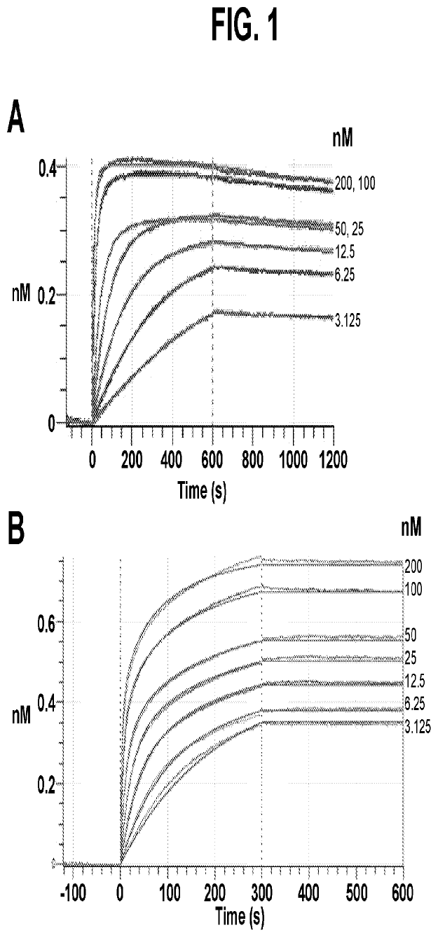

Anti-mCD117 antibody ACK2 and anti-mCD110 AMM2 are commercially available as rat immunoglobulins. The antibody variable domain sequences were obtained through in-solution endoproteinase digestion followed by liquid chromatography tandem mass spectrometry and data analysis. Murine IgG2a versions of the antibodies were generated by fusing the variable domains with murine heavy and light chain constant regions. Murine IgG2a_N297A versions of the antibodies were generated by mutagenesis of Asn in the murine Fc that is cognate to the human Fc Asn at position 297, to Ala. Chimeric antibodies were transiently produced from CHO cells, purified, and confirmed to bind to their respective murine antigens in binding assays using biolayer interferometry (ForteBio Octet) as shown in FIG. 1. Recombinant mCD110-ECD-H6 or mCD117-ECD-H6 was captured on sensor tips, which were transferred to solutions of anti-mCD110 mIgG2a or anti-mCD117 mIgG2a at the following concentrations: 200 nM, 100 nM, 50 nM, 25 nM, 12.5 nM, 6.25 nM, 3.125 nM, and 0 nM. After association of the antibodies, sensor tips were transferred to buffer alone to assess antibody dissociation over time. Association and dissociation curves were computed for each concentration level.

### Conditioning and Transplantation

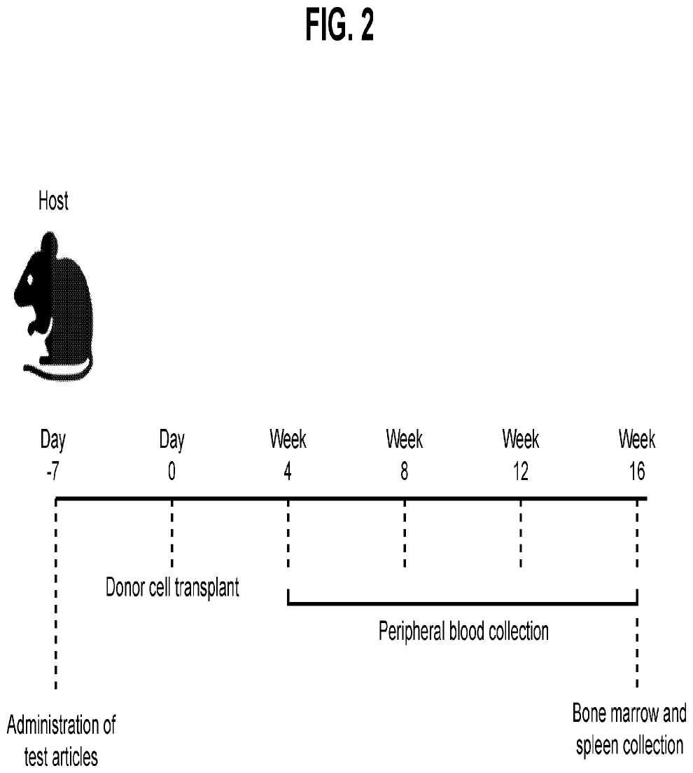

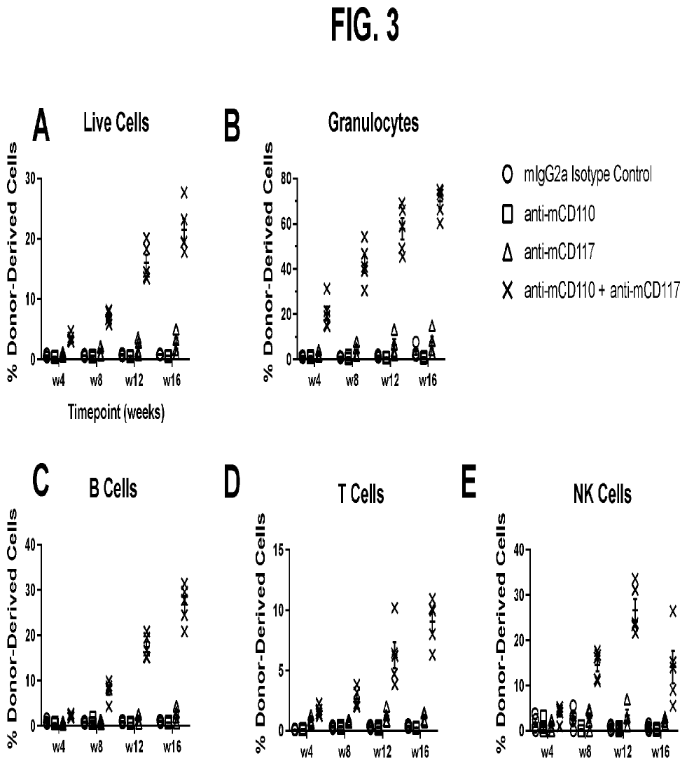

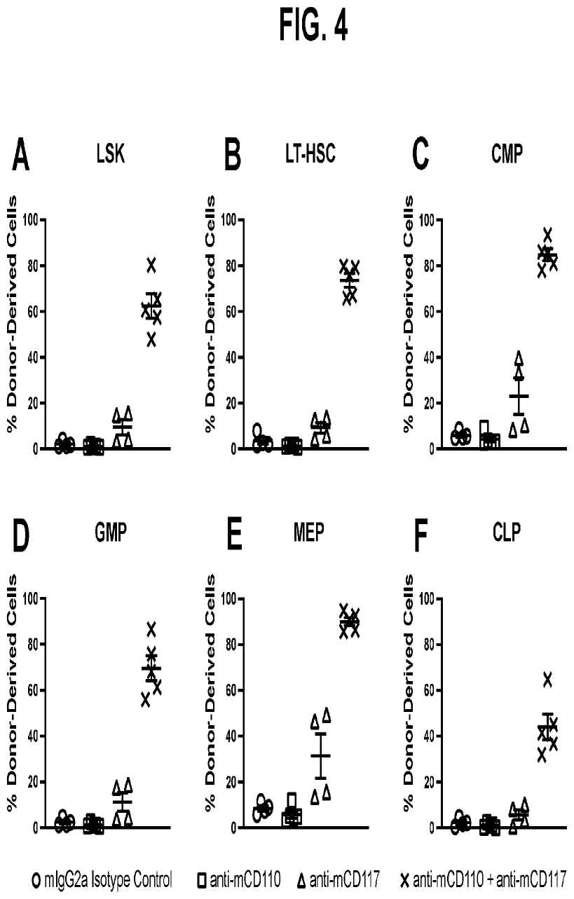

8-10 week old B6.SJL-Ptprc^{a} Pepc^{b}/BoyJ (“B6 CD45.1”) mice were treated with respective antibody regimens per study design in FIG. 2 on Day-7 with respect to bone marrow transplant. B6 CD45.1 mice were intravenously injected with 25 mg/kg of mIgG2a isotype control antibody, 25 mg/kg anti-mCD117 mIgG2a antibody, 25 mg/kg anti-mCD110 mIgG2a antibody, or both 25 mg/kg anti-mCD117 antibody and 25 mg/kg anti-mCD110 antibody. Animals were intravenously injected with 800,000 lineage-negative (“Lin”) donor cells isolated from C57BL/6J donor bone marrow cells. Chimerism results for this procedure are shown in FIG. 3 and FIG. 4.

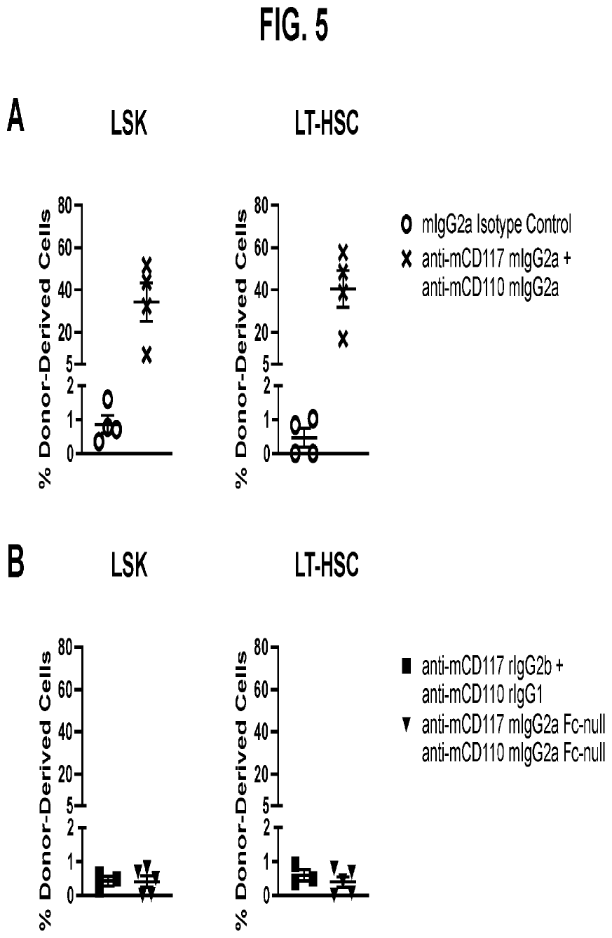

8-10 week B6 mice were intravenously injected with either A) 50 mg/kg mIgG2a isotype control antibody or a combination of 25 mg/kg anti-mCD117 mIgG2a antibody and 2.5 mg/kg anti-mCD110 mIgG2a antibody or with B)25 mg/kg ACK2 (anti-mCD117 rIgG2b antibody) and 5 mg/kg AMM2 (anti-mCD110 rIgGl antibody) or 25 mg/kg anti-mCD117 mIgG2a Fc-null antibody and 5 mg/kg anti-mCD110 mIgG2a Fc-null antibody on Day-7, and bone marrow transplant was performed on Day 0. Chimerism results for this procedure are shown in FIG. 5.

### Donor Cell Isolation

To isolate donor cells used in FIGS. 3 & 4, 8-10 week old B6 mice were euthanized and femora, tibiae, humeri, hips and vertebrae were collected. Bones were crushed to isolate bone marrow, followed by RBC lysis using Gibco ACK Lysing Buffer on ice for 7 minutes. Lineage negative cells were collected using Direct Lineage Cell Depletion kit (Miltenyi Biotec) in accordance with manufacturer's instructions.

To isolate donor cells used in FIG. 5, 8-10 week old B6 CD45.1 mice were euthanized and femora, tibiae, humeri, hips and vertebrae were collected. Bones were crushed to isolate bone marrow, followed by RBC lysis using Gibco ACK Lysing Buffer on ice for 7 minutes. Lineage negative cells were collected using Direct Lineage Cell Depletion kit (Miltenyi Biotec) in accordance with manufacturer's instructions.

### Co-Expression Analysis and Receptor Qantitation

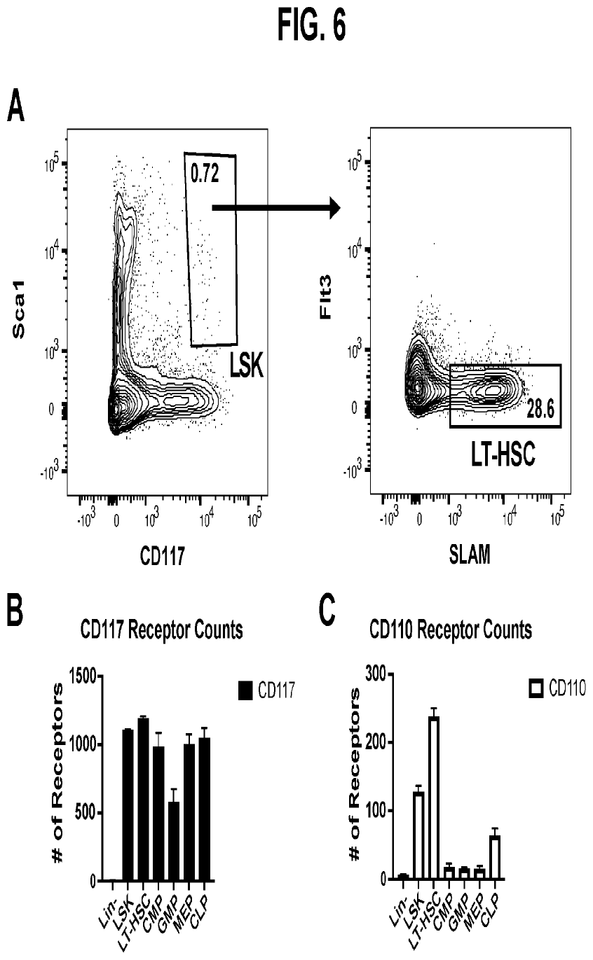

Naïve B6 mice were euthanized and femora and tibiae were collected. Bone marrow was extracted from bones via centrifugation, followed by RBC lysis using Gibco ACK Lysing Buffer on ice for 7 minutes. Cells were stained using fluorescent antibodies specific to the following targets: Flt3, CD117, CD34, CD127, Lineage (“Lin”: CD3e, Gr-1, CD11b, B220, TER119), Sca1, CD16/32, SLAM, and CD110. Samples were analyzed on the BD Fortessa X-20 cytometer alongside BangsLabs MESF AF647 Ladder, enabling quantitation of CD117 and CD110 receptors on HSPC populations (FIG. 6).

Fresh human bone marrow aspirates were processed by ficoll density gradient within 24 hours of collection. The resulting bone marrow mononuclear cells (BMMC) were stained with antibody panels and analyzed on a BD Fortessa X-20 cytometer.

The human antibody panel consisted of the following antibody/clone/fluorophore combinations: CD34(561)-APC, lineage dump CD3(UCHT1)/CD14(HCD14)/CD16(3G8)/CD19(HIB19)/CD20(2H7)/CD56(HCD56)-FITC, CD90(5E10)- BV421, CD45RA(HI100)-BV605, CD38(HIT2)-PE-Cy7, CD49f(GoH3)-BV510, CD110(1.6.1)-PE or CD117(104D2)-PE along with the viability stain FVS780-APC-Cy7.

### Chimerism Analysis

Mononuclear cells were collected from peripheral blood HetaSep (Stemcell technologies) followed by RBC lysis using Gibco ACK Lysing Buffer. To observe myeloid and lymphoid chimerism in peripheral blood, cells were stained using fluorescent antibodies specific to the following targets: CD19, CD11b, Ter119, CD45.2, NK1.1, Gr-1, CD45.1, and CD3. Dead cells were labeled using a fluorescent viability dye. Samples were analyzed on the BD Fortessa X-20 cytometer.

To assess chimerism of bone marrow cells, animals were euthanized, followed by collection of femora and tibiac. Bone marrow was isolated from bones via centrifugation, followed by RBC lysis using Gibco ACK Lysing Buffer. Bone marrow cells were isolated from naïve C57BL/6J mice and stained using fluorescent antibodies specific to the following targets: Flt3, CD117, CD34, CD127, Lineage (“Lin”: CD3e, Gr-1, CD11b, B220, TER119), Scal, CD16/32, SLAM, CD110. Dead cells were labeled using a fluorescent viability dye. Samples were analyzed on the BD Fortessa X-20 cytometer.

### Results

Wild-type B6 CD45.1 mice were treated with effector-competent 25 mg/kg anti-murine CD117 (mCD117) mIgG2a and 25 mg/kg anti-murine CD110 (mCD110) mIgG2a. 7 days post treatment animals were transplanted with 800,000 lineage depleted bone marrow cells from C57B16 mice that can distinguish a syngeneic transplant since they only differ by the CD45 allele (FIG. 2). Donor cell chimerism in peripheral blood was examined at 4, 8 12, and 16 weeks post-transplant. As shown in FIG. 3, while mCD117 mIgG2a alone resulted in 10% engraftment and mCD110 mIgG2a alone did not result in any engraftment, co-administration of both antibodies resulted in synergistic engraftment, as indicated by robust peripheral blood myeloid chimerism (Mac-1+Gr-1+ cells; FIG. 3B), B cell chimerism (CD19+ cells; FIG. 3C), T cell chimerism (CD3+ cells; FIG. 3D), and NK cell chimerism (NK1.1+ cells; FIG. 3E) that increased over time. These results demonstrate that an antibody-based conditioning regimen that simultaneously targets CD110 and CD117 results in a synergistic response enabling stable engraftment of donor HSPCs and multilineage hematopoietic reconstitution therefrom.

The use of monoclonal antibodies for effective conditioning of the bone marrow niche has been reported with anti-mCD117 or anti-mCD110 antibodies previously, but only ever in combination with chemotherapy and with regimens requiring multi-day dosing prior to donor stem cell transplant. For example, the rat anti-murine CD117 antibody ACK2 has been combined with 5-Azacytidine (AZA), a hypomethylating chemotherapeutic agent, to enable engraftment in murine bone marrow transplant models. This conditioning regimen requires six consecutive days of 5-Azacytadine administration (see e.g. Bankova et al., Blood Adv 5, 19 (2021)). Rat anti-murine CD110 antibody AMM2 has been combined with the chemotherapeutic agent 5-fluorouracil (5-FU) to enable engraftment (see e.g. Arai et al., Ann. N.Y. Acad Sci, 1176 (2009) and Yoshihara et al., Cell Stem Cell, 1 (2007). This is in contrast to the methods provided herein of combining two monoclonal antibodies, targeting CD117 and CD110, respectively, in a single dose with no use of chemotherapeutic agent, which avoids hazards to the recipient associated with genotoxic conditioning.

In the prior art examples cited above, the extent of chimerism observed with antibody-chemotherapy combinations was highly variable. The ACK2-AZA combination resulted in 30-60% bone marrow chimerism while the AMM2-5-FU combination resulted in 6% chimerism. Notably, the results provided herein demonstrating up to 60-80% chimerism upon the single co-administration of anti-CD117 and anti-CD110 antibodies in the absence of chemotherapy are surprising and unexpected based on the low activity of each individual antibody on its own. As shown in FIG. 4, no engraftment is observed with anti-CD110 administration alone (consistent with results reported by Arai et al., Ann. N.Y. Acad Sci, 1176 (2009)), and only 1-10% engraftment is observed with anti-CD117 administration alone (consistent with results reported by Bankova et al., Blood Adv 5, 19 (2021)). Arai et al. (Ann. N.Y. Acad Sci, 1176 (2009)) proposed that combination of ACK2 and AMM2 could achieve some level of engraftment based on the differential effects of ACK2 and AMM2 on different subpopulations (cycling cells versus quiescent cells, respectively) within the HSC population. In such a scenario, the effects on engraftment from combining ACK2 and AMM2 would be expected to be, at best, additive, expanding the types of cells (cycling and quiescent) that are targeted by these antibodies. Accordingly, our demonstration of synergistic engraftment when simultaneously targeting CD117 and CD110 with antibodies alone is unpredicted when taken in view of the prior art.

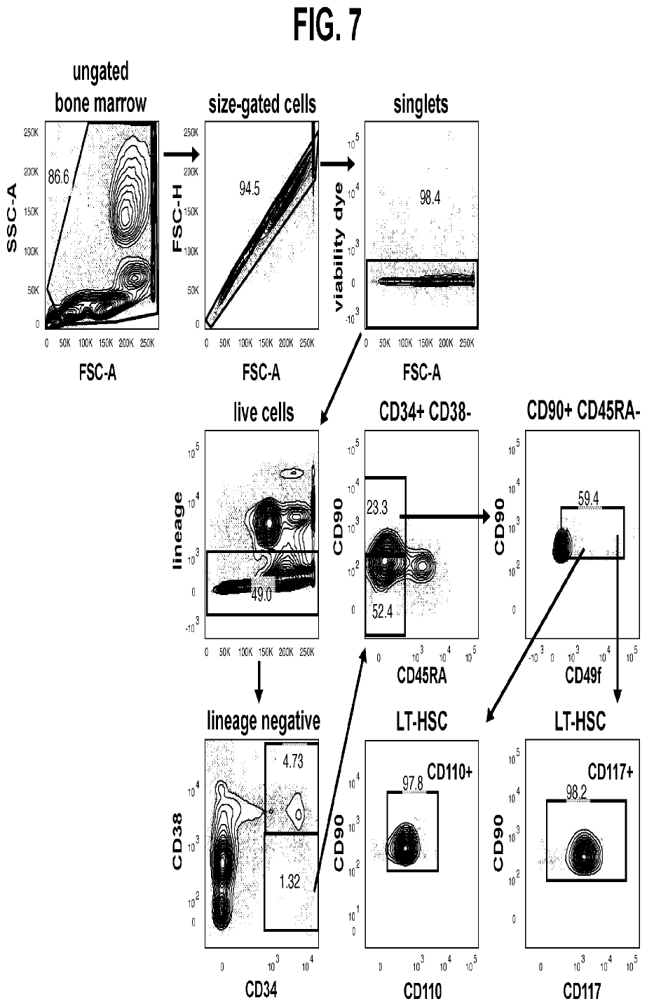

The unexpectedly superior chimerism shown in FIG. 4 is supported by additional results provided in FIG. 6 that demonstrate that CD110 and CD117 do not mark different truly long-term HSC cell populations. As shown in FIGS. 6B and 6C, the highest expression of both CD110 and CD117 was observed in LT-HSCs (Lin-CD117+Sca-1+Slam+Flt3-cells, FIG. 6A). Similarly CD110 and CD117 are co-expressed in human bone marrow LT-HSCs (Lin-CD34+CD38-CD45RA-CD90+CD49f+, FIG. 7).

Further, it was observed that the combination of ACK2 and AMM2 (Arai, 2009) does not actually lead to meaningful engraftment, as evidenced by the low percentage of donor-derived cells in recipient bone marrow 16 weeks post-transplant (FIG. 5B). ACK2 is a rat antibody to murine CD117 with an IgG2b isotype, while AMM2 is a rat antibody to murine CD110 with an IgG1 isotype. The synergistic effects shown in FIG. 3, 4, 5A with anti-CD117 and CD110 antibodies were observed when the Fc regions of ACK2 and AMM2 were modified from rat to murine isotype IgG2a. To assess the role of effector function in these versions, and since murine IgG2a is fully effector competent, we reengineered ACK2 and AMM2 as murine IgG2a antibodies rendered effector-incompetent by a previously described mutation in the Fc region (N297A) which removes potential glycosylation sites and may reduce Fc receptor binding (see, e.g., Shields R L et al., (2001) J Biol Chem 276:6591-604. The combination of effector-null anti-CD117 and anti-CD110 antibodies did not lead to synergistic donor cell engraftment, and instead yielded engraftment below 1%, similar to that observed with the combination of ACK2 and AMM2 (FIG. 5B)

Prior results showing engraftment using the ACK2-AZA regimen required ACK2administration prior to AZA treatment and was likely dependent on ACK2′s ability to block SCF from binding CD117, since co-administration of SCF or the use of another anti-murine CD117 antibody (2B8) with only partial ability to block SCF, negatively impacted LT-HSC depletion and/or subsequent engraftment (Bankova et al., Blood Adv 5, 19 (2021)). Our observation that only effector-competent versions of anti-CD110 and anti-CD117 antibodies combined to achieve synergistic preconditioning, while effector-null versions did not lead to durable donor cell engraftment, suggests a different mechanism of action compared to the antibody-chemotherapy regimens previously described. It has also been posited (Arai, et al., Ann. N.Y. Acad Sci, 1176 (2009)) that the lack of observed single agent effect with AMM2 alone could be due to residual circulating antibody negatively affecting the post-BMT expansion of donor cells. Our observation that synergistic, durable long term chimerism is achieved with the combination of anti-CD117 and anti-CD110 antibodies is counter to that supposition and is thus both novel and unexpected.

In summary, we describe a novel antibody-based conditioning regimen that simultaneously targets CD110 and CD117 co-expressed on HSPCs and LT-HSCs that results in a synergistic response enabling stable engraftment of donor HSPCs and multilineage hematopoietic reconstitution therefrom.

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

What is claimed:

1. A method of hematopoietic stem cell engraftment in a subject in need thereof, the method comprising:
a. depleting endogenous hematopoietic stem cells in the subject by administering to the subject a pharmaceutical composition comprising:
i. a first targeting moiety that specifically binds CD117; and
ii. a second targeting moiety that specifically binds CD110; and
b. administering exogenous hematopoietic stem cells to the subject;
wherein administration of the pharmaceutical composition mediates engraftment of the exogenous hematopoietic stem cells resulting in multi-lineage hematopoietic reconstitution in the subject.

2. The method of claim 1, wherein the first and second moiety bind hematopoietic stem cells co-expressing CD117 and CD110.

3. The method of claim 1 or 2, wherein the first targeting moiety comprises an isolated antibody or an antigen-binding fragment thereof that specifically binds CD117.

4. The method of claim 3, wherein the isolated antibody or antigen-binding fragment thereof that specifically binds CD117 functionally disrupts signaling between Stem Cell Factor (SCF) and CD117 and/or mediates clearance of CD117 expressing cells via Fc effector function.

5. The method of any one of claims 1 to 4, wherein the second targeting moiety comprises an isolated antibody, or an antigen-binding fragment thereof, that specifically binds CD110.

6. The method of claim 5, wherein the isolated antibody or antigen-binding fragment thereof that specifically binds CD110 functionally disrupts signaling between Thrombopoietin (TPO) and CD110 and/or mediates clearance of CD110 expressing cells via Fc effector function.

7. The method of any one of claims 3 to 6, wherein the isolated antibody of the first and/or second targeting moiety is a monoclonal antibody.

8. The method of any one of claims 3 to 6, wherein the antigen binding fragment of the first and/or second targeting moiety is selected from the group consisting of a Fv fragment, Fab fragment, F(ab′)2 fragment, Fab′ fragment, scFv (sFv) fragment, scFv-Fc fragment, single-chain Fvs (scFv), single-chain antibody, disulfide-linked Fvs (dsFv), fragments comprising either a VL or VH domain, a heavy chain antibody (hcAb), a single domain antibody (sdAb), a minibody, and a variable domain derived from camelid heavy chain antibodies (VHH or nanobody).

9. The method of any one of claims 1 to 8, wherein both the first targeting moiety and the second targeting moiety are comprised on the same antibody or antigen binding fragment thereof.

10. The method of claim 9, wherein the antibody or antigen binding fragment thereof is selected from the group consisting of a diabody, diabody-Fc, single-chain diabody, tandem diabody (Tandab's), tandem scFv, tandem scFv-scFc, tandem di-scFvs, tandem tri-scFvs, multivalent antibody, bivalent or bispecific single chain variable fragment, bispecific IgG and Fab-IgG bispecific.

11. The method of any one of claims 3 to 10, wherein the isolated antibody or antigen binding fragment of the first and/or second targeting moiety comprises an Fc region capable of binding the neonatal Fc receptor (FcRn) of the subject.

12. The method of any one of claims 3 to 11, wherein the isolated antibody or antigen binding fragment of the first and/or second targeting moiety is chimeric, humanized, or human.

13. The method of any one of claims 3 to 12, wherein the isolated antibody or antigen binding fragment of the first and/or second targeting moiety comprises a human Fc region.

14. A method of hematopoietic stem cell engraftment in a subject in need thereof, the method comprising:
a. depleting endogenous hematopoietic stem cells in the subject by co-administering to the subject:
i. an effective amount of a first isolated antibody, or an antigen-binding fragment thereof, that specifically binds CD117; and
ii. an effective amount of a second isolated antibody, or an antigen-binding fragment thereof, that specifically binds CD110; and
b. administering exogenous hematopoietic stem cells to the subject;
wherein co-administration of the effective amounts of the first and second antibodies, or fragments thereof, synergistically mediates engraftment of the exogenous hematopoietic stem cells resulting in multi-lineage hematopoietic reconstitution in the subject.

15. The method of claim 14, wherein the first and second isolated antibody, or antigen binding fragments thereof, bind hematopoietic stem cells co-expressing CD117 and CD110.

16. The method of claim 14 or 15, wherein the first isolated antibody, or antigen-binding fragment thereof, functionally disrupts signaling between Stem Cell Factor (SCF) and CD117 and/or mediates clearance of CD117 expressing cells via Fc effector function.

17. The method of any one of claims 14 to 16, wherein the second isolated antibody, or antigen-binding fragment thereof, functionally disrupts signaling between Thrombopoietin (TPO) and CD110 and/or mediates clearance of CD110 expressing cells via Fc effector function.

18. The method of any one of claims 14 to 17, wherein the first isolated antibody and/or the second isolated antibody is a monoclonal antibody.

19. The method of any one of claims 14 to 17, wherein the first isolated antibody and/or the second isolated antibody is a bispecific antibody.

20. The method of any one of claims 14 to 19, wherein the antigen binding fragment that specifically binds CD117 is selected from the group consisting of a Fv fragment, Fab fragment, F(ab′)2 fragment, Fab′ fragment, scFv (sFv) fragment, scFv-Fc fragment and nanobody fragment.

21. The method of any one of claims 14 to 20, wherein the antigen binding fragment that specifically binds CD110 is selected from the group consisting of a Fv fragment, Fab fragment, F(ab′)2 fragment, Fab′ fragment, scFv (sFv) fragment, scFv-Fc fragment, and nanobody fragment.

22. The method of any one of claims 14 to 21, wherein the Fc region of the first isolated antibody and/or the second isolated antibody is capable of binding the neonatal Fc receptor (FcRn) of the subject.

23. The method of any one of claims 14 to 22, wherein the first isolated antibody or antigen-binding fragment thereof and/or the second isolated antibody or antigen-binding fragment thereof is chimeric, humanized, or human.

24. The method of any one of claims 14 to 23, wherein the first isolated antibody or antigen-binding fragment thereof and/or the second isolated antibody or antigen-binding fragment thereof comprises a human Fc region.

25. The method of any one of claims 1 to 24, wherein the subject is human.

26. The method of any one of claims 3 to 25, wherein the antibody or antigen-binding fragment thereof that specifically binds CD117 and/or the antibody or antigen-binding fragment thereof that specifically binds CD110 is conjugated to a toxin.

27. The method of claim 26, wherein the toxin is selected from the group consisting of saporins, saporin derivatives, ricin, abrin, gelonin, momordin, apitoxin, shiga toxins, shiga-like toxins, T-2 mycotoxin, diphtheria toxin, busulfan, pseudomonas exotoxin A, Ricin A chain derivatives, trichosanthin, luffin toxin, maytansine, amatoxin, mechlorethamine, cyclophosphamide, ethylenimine, methylmelamine, methotrexate, fluorouracil, floxuridine, cytarabine, mercaptopurine, azathioprine, thioguanine, fludarabine phosphate, cladribine, dolastatin, auristatin, auristatin E, auristatin F, MMAF, MMAE, MMAD, DMAF, or DMAE, maytansine, DM1 or DM4, duocarmycin, calicheamicin, pyrrolobenzodiazepine, exatecan, and any combination thereof.

28. The method of any one of claims 1 to 27, further comprising monitoring the subject for depletion of endogenous hematopoietic stem cells prior to administering exogenous hematopoietic stem cells.

29. The method of any one of claims 1 to 28, wherein the exogenous hematopoietic stem cells are administered to the subject after the first and second isolated antibodies, or antigen-binding fragment(s) thereof, have substantially cleared from the blood of the subject.

30. The method of any one of claims 1 to 28, wherein the administering of exogenous hematopoietic stem cells to the subject occurs within 3, 5, 7 or 10 days of co-administering the first and second isolated antibodies, or antigen-binding fragment(s) thereof, to the subject.

31. The method of any one of claims 1 to 30, wherein the exogenous hematopoietic stem cells are allogeneic hematopoietic stem cells.

32. The method of any one of claims 1 to 30, wherein the exogenous hematopoietic stem cells are autologous hematopoietic stem cells.

33. The method of any one of claims 1 to 32, wherein the exogenous hematopoietic stem cells comprise CD34+ hematopoietic stem and progenitor cells (HSPCs).

34. The method of claim 33, wherein the CD34+ HSPCs comprise CD34+/CD38−/CD90+ HSPCs.

35. The method of claim 134 wherein the CD34+ HSPCs comprise CD34+/CD38−/CD90+/CD45RA-HSPCs.

36. The method of any one of claims 1 to 35, further comprising one or more of the following steps:
a. collecting a population of hematopoietic stem cells from the subject prior to depletion;
b. culturing the collected population of hematopoietic stem cells; and
c. cryopreserving the collected population of hematopoietic stem cells.

37. The method of claim 36, wherein collecting the population of hematopoietic stem cells from the subject comprises one or more of the following steps:
a. mobilizing the population of hematopoietic stem cells; and
b. collecting the population of hematopoietic stem cells by apheresis.

38. The method of any one of claims 1 to 37, wherein the exogenous hematopoietic stem cells are genetically modified.

39. The method of claim 38, wherein the exogenous hematopoietic stem cells are genetically modified using one or more components of a gene editing system.

40. The method of claim 39, wherein the one or more components of the gene editing system is selected from the group consisting of: (i) a CRISPR/Cas guide RNA, (ii) a DNA molecule encoding a CRISPR/Cas guide RNA, (iii) a nucleic acid molecule encoding a CRISPR/Cas RNA-guided polypeptide, (iv) a CRISPR/Cas RNA-guided polypeptide, (v) a CRISPR/Cas guide RNA complexed with a CRISPR/Cas RNA-guided polypeptide, (vi) a nucleic acid molecule encoding a zinc finger protein (ZFP), (vii) a ZFP, (viii) a nucleic acid molecule encoding a transcription activator-like effector (TALE) protein, (ix) a TALE protein, and (x) a DNA donor polynucleotide.

41. The method of claim 40, wherein the CRISPR/Cas RNA-guided polypeptide is a base editor or a prime editor.

42. The method of claim 39, wherein the one or more components of the gene editing system comprises a nuclease capable of generating a double-strand break within a gene locus of a cell.

43. The method of claim 42, wherein the one or more components of the gene editing system further comprises a DNA donor polynucleotide.

44. The method of claim 43, wherein the DNA donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR).

45. The method of claim 39, wherein the gene editing system comprises a CRISPR nuclease and a single guide RNA (sgRNA) capable of hybridizing to a target sequence within the gene locus, wherein the sgRNA guides the CRISPR nuclease to the target sequence.

46. The method of claim 45, wherein the CRISPR nuclease is a Cas protein.

47. The method of claim 46, wherein the Cas protein is Cas9 or a high-fidelity variant thereof.

48. The method of any one of claims 45 to 47, wherein the sgRNA and the CRISPR nuclease are formed in a ribonucleoprotein (RNP) complex.

49. The method of any one of claims 45 to 48, wherein the sgRNA comprises one or more chemically modified nucleotides.

50. The method of claim 49, wherein the modified nucleotide is selected from the group consisting of: a 2′-O-methyl nucleotide, a 2′-O-methyl 3′-phosphorothioate nucleotide, and a 2′-O-methyl 3′-thioPACE nucleotide.

51. The method of claim 49 or 50, wherein a 5′ end, a 3′ end, or a combination thereof of the modified sgRNA comprises a modified nucleotide.

52. The method of claim 51, further comprising contacting the population of stem cells with an AAV vector comprising a donor polynucleotide sequence.

53. The method of any one of claims 38 to 52, wherein the genetic modification corrects a gene mutation, replaces a mutant allele with a wild-type allele, or inserts a nucleic acid sequence encoding a therapeutic protein

54. The method of any one of claims 1 to 53, wherein the subject suffers from a disease.

55. The method of claim 54, wherein the disease is a hemoglobinopathy.

56. The method of claim 55, wherein the hemoglobinopathy is selected from the group consisting of sickle cell disease, α-thalassemia, β-thalassemia, and δ-thalassemia.

57. The method of any one of claims 1 to 56, wherein the engraftment occurs in the absence of myeloablative conditioning.

58. A method of depleting endogenous hematopoietic stem cells in a subject in need thereof, the method comprising administering to the subject a pharmaceutical composition comprising:
a. a first targeting moiety that specifically binds CD117; and
b. a second targeting moiety that specifically binds CD110.

59. The method of claim 58, wherein administration of the pharmaceutical composition mediates depletion of the exogenous hematopoietic stem cells in the subject. 60 The method of claim 58 or 59, wherein the first and second moiety bind hematopoietic stem cells co-expressing CD117 and CD110.

61. The method of any one of claims 58 to 60, wherein the first targeting moiety comprises an isolated antibody or an antigen-binding fragment thereof that specifically binds CD117.

62. The method of any one of claims 58 to 61, wherein the targeting moiety that specifically binds CD117 functionally disrupts signaling between Stem Cell Factor (SCF) and CD117 and/or mediates clearance of CD117 expressing cells via Fc effector function.

63. The method of any one of claims 58 to 62, wherein the second targeting moiety comprises an isolated antibody, or an antigen-binding fragment thereof, that specifically binds CD110.

64. The method of claim 63, wherein the isolated antibody or antigen-binding fragment thereof that specifically binds CD110 functionally disrupts signaling between Thrombopoietin (TPO) and CD110 and/or mediates clearance of CD110 expressing cells via Fc effector function.

65. The method of any one of claims 61 to 64, wherein the isolated antibody of the first and/or second targeting moiety is a monoclonal antibody.

66. The method of any one of claims 61 to 64, wherein the antigen binding fragment of the first and/or second targeting moiety is selected from the group consisting of a Fv fragment, Fab fragment, F(ab′)2 fragment, Fab′ fragment, scFv (sFv) fragment, scFv-Fc fragment, single-chain Fvs (scFv), single-chain antibody, disulfide-linked Fvs (dsFv), fragments comprising either a VL or VH domain, a heavy chain antibody (hcAb), a single domain antibody (sdAb), a minibody, and a variable domain derived from camelid heavy chain antibodies (VHH or nanobody).

67. The method of any one of claims 58 to 66, wherein both the first targeting moiety and the second targeting moiety are comprised on the same antibody or antigen binding fragment thereof.

68. The method of claim 67, wherein the antibody or antigen binding fragment thereof is selected from the group consisting of a diabody, diabody-Fc, single-chain diabody, tandem diabody (Tandab's), tandem scFv, tandem scFv-scFc, tandem di-scFvs, tandem tri-scFvs, multivalent antibody, bivalent or bispecific single chain variable fragment, bispecific IgG and Fab-IgG bispecific.

69. The method of any one of claims 61 to 68, wherein the isolated antibody or antigen binding fragment of the first and/or second targeting moiety comprises an Fc region capable of binding the neonatal Fc receptor (FcRn) of the subject.

70. The method of any one of claims 61 to 69, wherein the isolated antibody or antigen binding fragment of the first and/or second targeting moiety is chimeric, humanized, or human.

71. The method of any one of claims 61 to 70, wherein the isolated antibody or antigen binding fragment of the first and/or second targeting moiety comprises a human Fc region.

72. A method of depleting endogenous hematopoietic stem cells in a subject in need thereof, the method comprising co-administering to the subject:
a. an effective amount of a first isolated antibody, or an antigen-binding fragment thereof, that specifically binds CD117; and
b. an effective amount of a second isolated antibody, or an antigen-binding fragment thereof, that specifically binds CD110.

73. The method of claim 72, wherein co-administration of the effective amounts of the first and second antibodies, or fragments thereof, synergistically mediates depletion of the exogenous hematopoietic stem cells in the subject.

74. The method of claim 72 or 73, wherein the first and second isolated antibody, or antigen binding fragments thereof, bind hematopoietic stem cells co-expressing CD117 and CD110.

75. The method of any one of claim 2, 15, 60 or 74, wherein the hematopoietic stem cells co-expressing CD117 and CD110 are LT-HSCs.

76. The method of any one of claims 72 to 75, wherein the first isolated antibody, or antigen-binding fragment thereof, functionally disrupts signaling between Stem Cell Factor (SCF) and CD117 and/or mediates clearance of CD117 expressing cells via Fc effector function.

77. The method of any one of claims 72 to 76, wherein the second isolated antibody, or antigen-binding fragment thereof, functionally disrupts signaling between Thrombopoietin (TPO) and CD110 and/or mediates clearance of CD110 expressing cells via Fc effector function.

78. The method of any one of claims 72 to 77, wherein the first isolated antibody and/or the second isolated antibody is a monoclonal antibody.

79. The method of any one of claims 72 to 78, wherein the first isolated antibody and/or the second isolated antibody is a bispecific antibody.

80. The method of any one of claims 72 to 79, wherein the antigen binding fragment that specifically binds CD117 is selected from the group consisting of a Fv fragment, Fab fragment, F(ab′)2 fragment, Fab′ fragment, scFv (sFv) fragment, scFv-Fc fragment and nanobody fragment.

81. The method of any one of claims 72 to 80, wherein the antigen binding fragment that specifically binds CD110 is selected from the group consisting of a Fv fragment, Fab fragment, F(ab′)2 fragment, Fab′ fragment, scFv (sFv) fragment, scFv-Fc fragment, and nanobody fragment.

82. The method of any one of claims 72 to 81, wherein the Fc region of the first isolated antibody and/or the second isolated antibody is capable of binding the neonatal Fc receptor (FcRn) of the subject.

83. The method of any one of claims 72 to 82, wherein the first isolated antibody or antigen-binding fragment thereof and/or the second isolated antibody or antigen-binding fragment thereof is chimeric, humanized, or human.

84. The method of any one of claims 72 to 83, wherein the first isolated antibody or antigen-binding fragment thereof and/or the second isolated antibody or antigen-binding fragment thereof comprises a human Fc region.

85. The method of any one of claims 72 to 84, wherein the subject is human.

86. The method of any one of claims 72 to 85, wherein the antibody or antigen-binding fragment thereof that specifically binds CD117 and/or the antibody or antigen-binding fragment thereof that specifically binds CD110 is conjugated to a toxin.

87. The method of claim 86, wherein the toxin is selected from the group consisting of saporins, saporin derivatives, ricin, abrin, gelonin, momordin, apitoxin, shiga toxins, shiga-like toxins, T-2 mycotoxin, diphtheria toxin, busulfan, pseudomonas exotoxin A, Ricin A chain derivatives, trichosanthin, luffin toxin, maytansine, amatoxin, mechlorethamine, cyclophosphamide, ethylenimine, methylmelamine, methotrexate, fluorouracil, floxuridine, cytarabine, mercaptopurine, azathioprine, thioguanine, fludarabine phosphate, cladribine, dolastatin, auristatin, auristatin E, auristatin F, MMAF, MMAE, MMAD, DMAF, or DMAE, maytansine, DM1 or DM4, duocarmycin, calicheamicin, pyrrolobenzodiazepine, exatecan, and any combination thereof.
